(19) United States
(12) Patent Application Publication
(10) Pub. No.: US 2008/0153167 A1
(43) Pub. Date: Jun. 26, 2008

(54) In vivo amplification of neural progenitor cells

(75) Inventors: Peter Canoll, New York, NY (US); James Goldman, White Plains, NY (US); Marcela Assanah, East Elmhurst, NY (US); Sanja Ivkovic, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/821,086

(22) Filed: Jun. 21, 2007

Related U.S. Application Data
(60) Provisional application No. 60/815,488, filed on Jun. 21, 2006.

(51) Int. Cl. A61K 35/30; C12N 5/02; C12N 15/87; G01N 33/53; A61P 25/00; A01K 67/027; A61K 31/7088

(52) U.S. Cl. 435/464; 435/375; 514/44; 424/93.21; 800/10; 800/3

(57) ABSTRACT

The invention is directed to methods for in vivo amplification of neural progenitor cells using the proliferative environment of glial neoplasms in adult brain. The progenitor cells have the capacity to proliferate and differentiate into mature brain cells which can be used for cell replacement therapies. The invention also provides cell replacement therapy methods for treating central nervous system diseases or injuries, including multiple sclerosis, stroke, Alzheimer's disease and Parkinson's disease.

This application claims priority to U.S. Application No. 60/815,488, filed Jun. 21, 2006, which is hereby incorporated by reference in its entirety.

The invention disclosed herein was made with U.S. Government support under NIH Grant No. 5K08NSO45070-03 from the NINDS. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

The adult brain white matter contains a population of neural progenitor cells/stem cells that have the capacity to proliferate and give rise to mature brain cells, including astrocytes, oligodendrocytes and neurons. The normal fate of these progenitor cells is not known, but they may provide a source for cell replacement during brain injury or disease. For example, there is evidence that white matter progenitors contribute to the limited degree of remyelination that sometimes occurs during evolution of a multiple sclerosis lesion. However, it appears that their regenerative capacity is normally limited and the progenitors become depleted during the remitting and relapsing course of multiple sclerosis.

Currently, a major obstacle to developing an effective cell replacement therapy is generating sufficient numbers of these cells and therefore any method that can induce the proliferation/amplification of these progenitors is of great value. Previous studies have shown that a number of growth factors, including PDGF, FGF, GGF and EGF can induce progenitors to proliferate. Most of these studies have been done using in vitro cell culture systems using cells isolated from neonatal rodent brain. Much less is known about the regulation of proliferation, differentiation and survival of adult progenitor cells. Some studies have shown that isolated adult progenitors can be induced to proliferate to a limited extent in response to various growth factors in cell culture, but the conditions needed to generate large numbers of adult neural progenitor cells have not been identified.

### SUMMARY OF THE INVENTION

The invention provides for a method for amplifying neural progenitor cells, the method comprising administering a growth factor to a brain progenitor cell in the presence of brain tissue, so as to obtain recruitment of neural progenitor cells, thereby amplifying neural progenitor cells. In one embodiment, the method further comprises recovering the neural progenitor cells from the brain tissue. In another embodiment, the brain progenitor cell comprises an adult brain progenitor cell. In another embodiment, the brain progenitor cell comprises a neonatal progenitor cell. In another embodiment, brain tissue comprises white matter. In another embodiment, the brain tissue comprises a glial neoplasm.

In other embodiments of the methods of the invention, the growth factor comprises a PDGF, an FGF, a neuregulin, EGF, EGFR or any combination thereof. In one embodiment, the growth factor comprises PDGF-A. In another embodiment, the growth factor comprises PDGF-B. In another embodiment, the growth factor comprises FGF-2. In another embodiment, the growth factor comprises GGF-2.

In another embodiment, the growth factor is linked to a reporter molecule. In another embodiment, the growth factor is co-expressed with a reporter molecule. In another embodiment, co-expression is achieved by an internal ribosome entry site (IRES). in another embodiment, the reporter molecule comprises a fluorescent protein. In another embodiment, the reporter molecule comprises green fluorescent protein, yellow fluorescent protein, blue fluorescent protein or DsRed.

In one embodiment, expression of the growth factor is inducible. In another embodiment, expression is induced by tetracycline. In another embodiment, expression is induced by tamoxifen. Inducible expression can be achieved by using an inducible promoter. Non-limiting examples of inducible promoters are tetracycline inducible promoters (both “tet on” and “tet off” systems). Another non-limiting example is to use an inducible cre recombinase system (such as cre-ER).

In one embodiment of the invention, the administering comprises introducing a replication incompetent retrovirus encoding the growth factor. In another embodiment, the retrovirus is an amphoteric retrovirus.

In another embodiment, the administering comprises introducing a cell containing an isolated nucleotide sequence capable of expressing the growth factor, wherein the cell expresses the growth factor. In one embodiment, the cell is a neural progenitor cell.

The invention also provides a method for obtaining neural progenitor cells, the method comprising (a) infecting a brain progenitor cell with a replication incompetent retrovirus encoding a PDGF-B growth factor in the presence of brain tissue, and (b) recovering amplified neural progenitor cells from the brain tissue.

The invention provides a method for amplification of resident progenitor cells in a tissue or organ, the method comprising administering a growth factor to the tissue or organ, so as to obtain recruitment of resident progenitor cells in the tissue or organ. In one embodiment, the administration of the growth factor to the tissue or organ is in vivo. In another embodiment, the administration of the growth factor to the tissue or organ is in vitro. In another embodiment, the administering comprises introducing a nucleic acid encoding the growth factor. In another embodiment, the tissue or organ comprises brain, pancreas, liver, spinal cord, bone, heart, or kidney.

The invention provides method for treating a CNS injury or disease in a subject, the method comprising administering directly to the CNS of the subject a nucleic acid capable of expressing a growth factor, wherein expression of the growth factor causes recruitment of neural progenitor cells, thereby causing amplification of neural progenitor cells. In one embodiment, the method further comprises decreasing the expression of the growth factor, thereby decreasing the levels of growth factor in the brain, whereby the amplified neural progenitor cells will become differentiated. In another embodiment, the method further comprises, (a) recovering the neural progenitor cells from the subject; (b) differentiating the neural progenitor cells in vitro; and (c) returning the differentiated progenitor cells of step (b) to the CNS of the subject. In another embodiment, the CNS injury or disease comprises Alzheimer's disease, multiple sclerosis, Parkinson's disease, Huntington's disease, stroke, dementia, trauma or any combination thereof. In one embodiment, the nucleic acid comprises an inducible promoter operably linked to a nucleic acid sequence encoding the growth factor.

In one embodiment of the methods of the invention, recovering the progenitor cells comprises cell sorting. In another embodiment, the cell sorting comprises fluorescence activated cell sorting. In another embodiment, the cell sorting comprises immunodetection of a progenitor cell surface molecule. In another embodiment, the progenitor cell surface molecule is A2B5 surface ganglioside. Other examples of techniques that can be used to isolate cells include magnetic beads and immunopanning. In another embodiment, differentiating the progenitor cells comprises culturing under conditions which induce the formation of astrocytes, oligodendrocytes, neurons, or any combination thereof.

The invention provides a method for cell replacement therapy to treat an injury or disease in a subject, the method comprising (a) administering one or more growth factors directly to a tissue of the subject, so as to obtain recruitment of resident progenitor cells in the tissue; (b) recovering the progenitor cells from the subject; (c) differentiating the progenitor cells in vitro; and (d) returning the differentiated progenitor cells to the tissue of the subject. In one embodiment, the administering comprises intralesional, intraperitoneal, intramuscular, intratumoral or intravenous injection; infusion; liposome- or vector-mediated delivery; or topical, nasal, oral, ocular, otic delivery, or any combination thereof. In another embodiment, the administering is directly to the brain. In another embodiment, the differentiating comprises defined cell culture conditions, genetic engineering of the cells, or a combination thereof. In another embodiment, the tissue comprises brain, pancreas, liver, spinal cord, bone, heart, or kidney. For example, growth factor-free/serum-free basal media conditions allows many cells to differentiate along the oligodendrocyte lineage and some to differentiate along the neuronal lineage.

The invention provides a genetically modified animal, wherein the animal's brain has been infected with one or more amphoteric viral vectors, and wherein at least one vector comprises a nucleic acid encoding PDGF-B and PDGF-B is expressed therefrom in the animal's brain, thereby forming a tumor in the animal's brain. In one embodiment, at least one vector comprises a nucleic acid encoding a reporter molecule and the reporter molecule is expressed therefrom in the animal's brain.

The invention also provides a method for determining whether a test compound is capable of treating a brain tumor, the method comprising (a) introducing one or more amphoteric viral vectors into an animal's brain, wherein at least one vector comprises a nucleic acid encoding PDGF-B and PDGF-B is expressed therefrom in the animal's brain, thereby forming a tumor in the animal's brain; (b) administering an effective amount of the test compound to the animal of step (a); (c) measuring the growth of the tumor in the animal of step (a); and (d) comparing the measurement of tumor growth of step (b) to a measurement of tumor growth in an animal treated as in step (a) to which the test compound was not administered, wherein an arrest, delay, or reversal in tumor growth in the animal of step (a) indicates that the test compound is capable of treating a brain tumor.

The invention also provides a method for determining whether a test compound is capable of preventing a brain tumor, the method comprising (a) introducing one or more amphoteric viral vectors into an animal's brain, wherein at least one vector comprises a nucleic acid encoding PDGF-B and PDGF-B is expressed therefrom in the animal's brain; (b) administering an effective amount of the test compound to the animal of step (a), wherein the administering occurs before formation of a tumor in the animal's brain; (c) assessing brain tumor formation in the animal of step (a); and (d) comparing the assessment of tumor formation of step (b) to a measurement of tumor formation in an animal treated as in step (a) to which the test compound was not administered, wherein an absence of tumor formation in the animal of step (a) indicates that the test compound is capable of preventing a brain tumor.

The invention further provides a method for determining brain tumor recurrence following brain tumor treatment, the method comprising (a) introducing one or more amphoteric viral vectors into an animal's brain, wherein at least one vector comprises a nucleic acid encoding PDGF-B and PDGF-B is expressed therefrom in the animal's brain, and wherein at least one vector comprises a nucleic acid encoding a reporter molecule and the reporter molecule is expressed therefrom in the animal's brain, thereby forming a tumor in the animal's brain; (b) administering to the animal of step (a) one or more compounds in an effective amount to treat the tumor; and (d) detecting expression of the reporter molecule in the brain of the animal of step (a), wherein detection of the reporter molecule indicates that the tumor will recur. In one embodiment, PDGF-B and the reporter molecule are co-expressed from one vector. In another embodiment, co-expression is achieved by an internal ribosome entry site (IRES).

In the context of the invention, administration may be effected by transplantation; intralesional, intraperitoneal, intramuscular or intravenous injection; by infusion; or may involve liposome-mediated delivery; or topical, nasal, oral, anal, ocular or otic delivery.

In the practice of the method, administration may comprise daily, weekly, monthly or hourly administration, the precise frequency being subject to various variables such as age and condition of the subject, amount to be administered, half-life of the agent in the subject, area of the subject to which administration is desired and the like.

In connection with the method of this invention, a therapeutically effective amount of the inhibitor may include dosages which take into account the size and weight of the subject, the age of the subject, the severity of the symptoms, the method of delivery of the agent and the history of the symptoms in the subject.

### BRIEF DESCRIPTION OF THE FIGURES

FIGS. 1A-1E. PDGF overexpression induces the formation of malignant gliomas. Hematoxylin and eosin stains were performed on coronal sections of adult rat brains 14 dpi with pQ-GFP (FIGS. 1A and 1C) or PDGF-IRES-GFP (FIGS. 1B, 1D, and 1E). FIG. 1A: No tumors formed in brains injected with the control retrovirus, but a small area of reactive gliosis is seen around needle track (arrow). FIG. 1C: Higher magnification micrograph showing the injection site. FIG. 1B: Large infiltrative tumors with the histological features of human glioblastoma formed by 14 dpi with PDGF-IRES-GFP retrovirus. This section, 3 mm caudal from injection site, shows the tumor extending across the corpus callosum into the contralateral hemisphere. FIG. 1D: Higher magnification micrograph showing an area of pseudopalisading necrosis (N), a hallmark of glioma malignancy seen in human glioblastomas. FIG. 1E: Tumor cells crossing the corpus callosum (CC) and infiltrating the cortex (CX). Scale bar=2 mm in FIGS. 1A and 1B.

FIGS. 2A-2C. GFP expression reveals the distribution of retrovirally infected cells. Immunofluorescence analysis for GFP and SMA was performed on sections of tumor at 17 dpi with the PDGF-IRES-GFP retrovirus. FIG. 2A: Micrograph shows GFP+ cells (green) crossing the corpus callosum (CC) and invading the contralateral hemisphere. Hoechst stain (blue) shows increased cellular density in and around the main tumor mass. Note that only a subset of the cells is GFP+. FIG. 2B: GFP+ and GFP− cells are seen intermingled throughout the tumor, including in areas of pseudopalisading necrosis (N). FIG. 2C: SMA immunofluorescence (red) shows marked vascular proliferation with recruitment of perivascular smooth muscle cells. Scale bar=1 mm in FIG. 1A.

FIGS. 3A-3D. GFP+ and GFP− tumor cells express markers of glial progenitor cells. Double immunofluorescence analysis of tumors, 17 dpi with the PDGF-IRES-GFP retrovirus, shows that GFP (green) is expressed in only a subset of tumor cells whereas nestin (a′-a″), NG2 (b′-b″), and PDGFRα (c′-c″), each stained red, are expressed in the vast majority of GFP+ and GFP−tumor cells. (d′-d″) GFAP+/GFP− reactive astrocytes (red) are seen scattered throughout the tumor. Rare GFAP+/GFP+ cells were seen (less than 3%), however, the vast majority of the GFP+ cells do not express detectable levels of GFAP.

FIGS. 4A-4F. Quantitative analysis shows that the majority of cells in the PDGF driven tumors are GFP−progenitor cells. FIG. 4A: Double immunofluorescence of tumors, 17 dpi with PDGF-IRES-GFP, shows that the vast majority of tumor cells are olig2+(red), but only a subset of olig2+ cells are GFP+ (green). FIG. 4B: Double immunofluorescence for GFP (green) and Ki67 (red), a marker for cycling cells, shows that tumor cells are highly proliferative but only a subset of Ki67+ cells are GFP+. The triptych at the bottom of FIGS. 4A and 4B show green, red, and green/red overlay. FIG. 4C: Bar graph showing the number of total cells (identified by Hoechst nuclear staining), GFP+, olig2+, GFP+/olig2+, and GFP−/olig2+ cells per high-powered field. FIG. 4D: Bar graph showing the number of total, GFP+, ki67+, GFP+/ki67+, and GFP−/ki67+ cells per high-powered field. Data represents the mean +/−S.E.M. of multiple high-powered fields from 3 separate brain tumors. FIGS. 4E and 4F: Cells isolated from freshly dissected tumors (17 dpi with PDGF-IRES-GFP retrovirus), were immunostained with the progenitor cell marker A2B5 and analyzed by flow cytometry. FIG. 4E: Scatter plot from a single representative experiment showing the relative abundance of 4 populations of tumor cells: GFP+/A2B5+, GFP−/A2B5+, GFP+/A2B5−, and GFP−/A2B5−. FIG. 4F: Bar graph showing the percent of cells in each population. Data represents the mean +/−S.E.M. from 3 separate experiments.

FIGS. 5A-5F. Co-injection studies show that PDGF expressing cells recruit other progenitors to proliferate within the tumor. FIG. 5A: Low power micrograph showing a small collection of GFP+ cells (green) along the injection track (white arrow) at 17 dpi with pNIT-GFP. FIG. 5B: In contrast, a large tumor containing many GFP+ cells (green) and DsRed+ cells (red) are seen at 17 dpi with pNIT-GFP and PDGF-IRES-DsRed. Note that many GFP+ cells are seen crossing the corpus callosum (CC) into the contralateral hemisphere. FIG. 5C: Higher magnification micrograph of tumor showing red and green cells intermingled throughout the tumor, including areas of pseudopalisading necrosis (N). FIG. 5D: Cells isolated from tumors (19 dpi with pNIT-GFP and PDGF-IRES-DsRed) were FAC sorted into 4 populations: GFP−/DsRed−, GFP+/DsRed−, DsRed+/GFP−, and DsRed+/GFP+. 1.5×10⁶ GFP+/DsRed−cells and 2.8×10 ₅ DsRed+/GFP−cells were sorted from 2 pooled tumors in this representative experiment (the experiment has been repeated five times). FIG. 5E: In brains injected with pNIT-GFP (17 dpi), the GFP+ cells have a highly branched morphology and are negative for the proliferation marker Ki67 (labeling index of less than 5%). FIG. 5F: In tumors generated by co-infection of pNIT-GFP and PDGF-IRES-DsRed the GFP+ cells have an immature morphology and many are ki67+ (labeling index of 26%).

FIGS. 6A-6B. Two models that account for the heterogeneity of glioma cells. FIG. 6A: The cancer stem cell model states that gliomas contain a small subset of cells with stem cell like properties (dark circles). These cells have an inexhaustible capacity to self-renew and even a few cells are able to give rise to new tumor formation. The bulk of the tumor is composed of the tumor stem cells' more differentiated progeny (light circles). These cells may be capable of proliferating, at least transiently, but they have limited capacity to self-renew and are therefore unable to initiate new tumor growth. FIG. 6B: The progenitor recruitment model states that tumor initiating cells (dark circles) are capable of recruiting genetically normal progenitors (light circles) to proliferate via paracrine growth factor signaling (thick black arrows). The genetically normal progenitor cells look and behave like glioma cells while they are exposed to high concentrations of growth factors, but they may stop proliferating and differentiate if they are isolated from the mitogenic tumor environment. The two models are not mutually exclusive.

FIG. 7. PDGF retrovirus causes rapid tumor formation and morbidity. Kaplan-Meier survival curve showing the onset of tumor induced morbidity. Equal volumes and titers of pQ-GFP or PDGF-IRES-GFP were injected into the subcortical white matter of adult rats (6 rats in each group). All rats injected with the PDGF-IRES-GFP retrovirus showed signs of tumor-induced morbidity between 14 and 19 dpi. None of the rats injected with pQ-GFP showed any signs of tumor-induced morbidity at 35 dpi.

FIG. 8. Serial MRI studies shows tumor progression. MRI scans were performed on adult rats at 5, 10, and 17 dpi with the PDGF-IRES-GFP retrovirus. Here we show a representative series from one animal. No tumor is visible at 5 dpi. By 10 dpi a small tumor is seen at the injection site on post-contrast T1 weighted images. Tumor induced edema is visible on T2 images. At 17 dpi a large tumor is visible on post-contrast T1 image, and edema involves the entire hemisphere and part of the contralateral hemisphere as seen in the T2 weighted image.

FIGS. 9A-9B. VENN diagrams illustrating the relationship between the subpopulations of GFP+, olig2+, and Ki67+ cells in PDGF induced tumors. FIG. 9A: The red area represents olig2+/GFP−cells (61.7%). The yellow area represents GFP+/olig2+ cells (16.3%). The green area represents GFP+/olig2-cells (0.7%). The blue area represents GFP−/olig2-cells (21.3%). FIG. 9B: The red area represents Ki67+/GFP−cells (27.9%). The yellow area represents GFP+/Ki67+ cells (6.1%). The green area represents GFP+/Ki67-cells (10.9%). The blue area represents GFP−/Ki67-cells (55.1%). These values are derived from the quantitative analysis shown in FIGS. 4A-4F.

FIGS. 10A-10D. Adult white matter progenitors infected with PDGF-IRES-dsRed in vitro form malignant gliomas through autocrine and paracrine signaling. FIGS. 10A and 10B: Normal adult white matter progenitors were isolated and expanded for 5 days in vitro with B104 containing media. Proliferating progenitors were then infected with pNIT-GFP or PDGF-IRES-DsRed. At 2 dpi the cells were implanted into adult brains. Brains were analyzed at 20 dpi. FIG. 10A: pNIT-GFP infected cells injected alone remain close to the injection site and acquire more mature glial morphology. No tumors formed. FIG. 10B: When pNIT-GFP infected cells are co-injected with PDGF-IRES-DsRed infected cells a tumor forms by 20 dpi that is composed of a mixture of red and green cells. The green cells remained immature, migratory, and proliferative. FIGS. 10C and 10D: Adult white matter progenitors (expanded and infected as described above) were grown in culture for 10 dpi. FIG. 10C: pNIT-GFP infected cells stop proliferating and acquire a highly branched morphology. FIG. 10D: PDGF-IRES-DsRed infected cells retain a simple morphology and remain highly proliferative, forming large clusters of cells.

FIGS. 11A-11B. Olig2+/NG2+ adult progenitor cells.

FIG. 12. Self-inactivating bicistronic expression retroviral vector designed to express two target genes. PDGF-HA was inserted into MCS I; eGFP or DsRed was inserted into MCS II.

FIG. 13. Front and side views of rat brain.

FIG. 14. Retroviral labeled progenitor cells in adult rat white matter at 17 days post-injection (GFP—green; xki67—red).

FIG. 15. Expression of PDGF in rat brain 14 days post-injection.

FIGS. 16A-16B. The PDGF virus causes rapid expansion of the progenitor cells. (FIG. 16A: 6 dpi; FIG. 16B: 17 dpi)

FIGS. 17A-17B. Detection of Olig2 (red) and GFP (green) in adult rat brain 17 dpi (FIG. 17A: control; FIG. 17B: PDGF).

FIGS. 18A-18B. Detection of K167 (red) and GFP (green) in adult brain 17 dpi (FIG. 18A: control; FIG. 18B: PDGF).

FIG. 19. A2B5+ and O4+ cells from adult rat white matter and tumors.

FIGS. 20A-20B. Tumors composed of many red and green cells form by 17 dpi with pNIT-GFP and PDGF-IRES-dsRED (FIG. 20A). FAC sorting is used to separate the pNIT-GFP+ (normal progenitors) from PDGF-IRES-dsRED cells (FIG. 20B).

FIGS. 21A-21F. Isolated GFP+ cells differentiate into CC1+ oligodendrocytes and TUJ1+ neurons in vitro.

FIG. 22. Isolation from human tumors and adult white matter.

FIG. 23. Targeting growth factor signaling. Growth factor receptors, such as PDGFR and EGFR regulate cell migration, proliferation and survival by activating a number of cytoplasmic signaling pathways. This diagram shows a simplified representation of two of the best characterized signaling cascades, the PI3K-AKT and RAS-MAPK pathways. These signaling cascades involve a series of reversible phosphorylation events that regulate the association and enzymatic activity of the various signaling molecules. Also shown is a partial list of the small molecule inhibitors that have been shown to inhibit specific steps of these pathways.

FIGS. 24A-24B. Immunohistochemical analysis for phospho-S6 kinase. PDGF driven tumors were stained with a phospho-specific antibody against p70S6 kinase. FIG. 24A: High levels of staining are seen in the cytoplasm of tumor cells except in areas of pseudopalisading necrosis (N). FIG. 24B: Strong staining is seen in a subset of cells infiltrating the corpus callosum.

FIGS. 25A-25B. Time-lapse analysis of GFP+ cells migrating in a PDGF driven tumor. FIG. 25A shows double immunofluorescense analysis of a fixed section of a rat brain 10 dpi with the PDGF-IRES-GFP expressing retrovirus. At this time point a tumor (T) has formed on the side of the injection site and numerous GFP+ cells (green) are seen infiltrating the cortex (CX) and crossing the corpus callosum (CC). The section was also stained with SMA (red), showing large vessels in the meninges and ventricle. FIG. 25B shows the results of a 15 hour time-lapse experiment on a slice culture generated at 10 dpi. The slice was taken at the same level as the section shown in A. The green boxes show areas where the migratory paths (red lines) of the GFP+ cells were tracked using DIAS. The data is superimposed onto the last frame of the time-lapse. Note that the migratory paths of cells within the tumor (T) are shorter and less directed than the paths of cells migrating across the corpus callosum (CC). The yellow arrow points to the path of cell that migrated several hundred μm through the corpus callosum during the course of the time-lapse. The graph (B′) shows the speed of this cell over the course of the time-lapse. There are marked fluctuations in speed as the cells moves in a salutatory manner, with maximum speed of over 100 μm/hour.

FIG. 26. Dynamic analysis of cell migration in the PDGF driven gliomas. 300 μm thick slice cultures were prepared at 10 dpi with the PDGF expressing retrovirus. A large tumor had formed at the injection site many GFP+ cells were seen infiltrating the surrounding brain. Cells were monitored by time-lapse microscopy for 150 minutes. The micrographs show selected time points of a GFP+ cells infiltrating the overlying cortex. The cell has a long leading process that extends and retracts with rapid dynamics while the cell body moves in a saltatory manner, leaving behind a thin cytoplasmic tail.

FIG. 27. EGFR-GFP expressing cells disperse widely throughout the brain. Control CLE-GFP retrovirus or EGFR-GFP retrovirus (10⁵ CFU in 1 μl) was injected into the subcortical white matter of neonatal rats. At 15 weeks post-injection the animals were sacrificed and the distribution of GFP+ cells was mapped by fluorescence analysis of serial coronal sections though the brain, Note that the EGFR-GFP+ cells expanded greatly in number and migrated widely throughout the white matter. In contrast, the CLE-GFP+ cells remain few and were distributed relatively near the injection site.

FIG. 28. EGFR-GFP+ cells express PDGFRα. EGFR-GFP expressing retrovirus was injected into the neonatal rat brain. Immunofluorescence analysis at 15 wpi shows that EGFR-GFP (green) and PDGFRα (red) are expressed by the same cells. These cells also express NG2 and olig2 but do not express detectable levels of GFAP.

FIG. 29. EGFR-GFP accumulates in the leading process of migrating progenitor cells. 300 μM thick slice cultures were prepared from neonatal rat forebrains at 5 dpi with EGFR-GFP expressing retrovirus and cells were monitored by time-lapse microscopy. Frames show selected time points over 180 minutes duration. Note that the EGFR-GFP accumulates in the leading process (arrows) as the migrating cell turns and starts to migrate in the opposite direction.

FIGS. 30A-30C. EGFR-GFP is focally phosphorylated. Immunofluorescence analysis at 15 wpi with EGFR-GFP retrovirus shows an EGFR-GFP+ cell with punctate accumulations of EGFR-GFP (arrows) that co-localize with phosho-EGFR immunoreactivity.

FIG. 31. EGFR-GFP is distributed into both daughter cells during cell division. Time-lapse microscopy of a slice culture prepared at 5 dpi shows an EGFR-GFP+ cell dividing. Note that as the cell goes through cytokinesis the EGFR-GFP is distributed onto the surface of both daughter cells.

FIGS. 32A-32B. EGFR-GFP expressing cells form malignant brain tumors. EGFR-GFP expressing retrovirus was injected into the subcortical white matter of neonatal rats. Approximately one year after injection the animal developed signs of tumor induced morbidity. FIG. 32A is a low power micrograph showing the large tumor (T) that is composed predominantly of GFP+ cells (green). Many GFP+ cells are seen infiltrating the overlying cortex (CX). FIG. 32B is a high power micrograph showing GFP+ tumor cells surrounding a GFP− proliferative blood vessel (V). The nuclei are stained with DAPI (blue).

FIG. 33. Glioblastomas contains a massive proliferation of A2B5+ cells. Two grams of tissue from adult human white matter and glioblastomas were dissociated, stained for A2B5 (to label progenitor-like cells) and CD45 (to label microglia) and analyzed by flow cytometry. The scatter plots show the relative abundance of the different cell populations. Approximately 8×10³ A2B5+ cells were isolated from white matter and 4×10⁶ A2B5+ cells were isolated from the glioblastomas.

FIG. 34. Flow cytometry analysis of CD133+, A2B5+ and O4+ cell in an oligodendroglioma (left) and glioblastomas (right). Both tumors contain a prominent population of A2B5+ cells. Note that in the glioblastomas most of the CD133+ cells stain for A2B5 but not for O4. The low-grade oligodendroglioma has a higher percentage of O4+ cells and virtually no CD133+ cells.

FIG. 35. A2B5+ cells form diffusely infiltrating gliomas when injected into nude rats. The left panel is a low power micrograph showing a hematoxylin and eosin stain of the tumor (T) with cells infiltrating the cortex (cx), corpus callosum (cc) and striatum (s). The right panel is a high power micrograph showing the infiltrative edge of the tumor.

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

It is a discovery of the present invention that the majority of cells that comprise glial neoplasms are unmodified, normal, adult neural progenitor cells that have been recruited to the tumor via paracrine signaling. The invention provides methods to exploit the proliferative capacity of glial neoplasms to generate unprecedented numbers (millions) of progenitor cells in the adult brain which retain the capacity to differentiate and are useful for cell replacement therapy. Using the methods of the invention, millions of progenitor cells can be generated from one rat brain, as opposed to the hundreds or thousands of cells generated by other reported methods. Additionally, the use of adult progenitor/stem cells in the methods of the present invention reduces likelihood of ethical concerns that are frequently associated with the use of embryonic stem cells.

The methods of the invention use a retrovirus that expresses high levels of platelet derived growth factor-B (PDGF-B) and reporter genes (for example, GFP or DsRed). In contrast to previous glioma models, the invention provides for the use of an amphoteric retrovirus. An amphoteric retrovirus is capable of infecting any cycling cell. When the virus (10⁵ cfu) is injected into the white matter of adult rodent brains, it infects resident progenitor cells and induces proliferation of progenitors via autocrine and paracrine signaling. Because the cells that are producing the PDGF-B are also proliferating via autocrine stimulation, the concentration of PDGF-B continues to rise as the cell population expands. By 17 days post-injection (dpi), the concentration of PDGF is more than 3000 fold higher than that of normal brain and there is a mass of cells composed predominantly of non-infected neural progenitor cells that have been recruited via paracrine signaling. When the recruited progenitors are removed from the mitogenic environment of the tumor (e.g., isolated to an in vitro setting, or growth factor stimulus is turned off in vivo), they stop proliferating and are capable of differentiating into astrocytes, oligodendrocytes or neurons.

Amplification of Neural Progenitor Cells

Glioma cells closely resemble glial progenitors in their dependence on growth factor stimulation to drive their migration, proliferation and survival. The present invention provides that a retrovirus expressing a growth factor, for example PDGF, can be used to create a highly proliferative environment in vivo to amplify neural progenitors in the adult brain.

Growth Factors

Previous studies show that several growth factors, including PDGF, induce proliferation of adult neural progenitor cells. Most of these studies have been done using in vitro cell culture systems using cells isolated from neonatal rodent brain. Generally, the findings of in vitro studies are indicative of in vivo biological processes. In vitro studies show that treating adult progenitors with PDGF induces a limited proliferative response (Shi et al. 1998; Wolswijk and Noble 1992; Wolswijk et al. 1991). Therefore, based on these in vitro experiments, exposing adult progenitors within the brain to PDGF would not be expected to be sufficient induce a massive proliferation of progenitor cells.

It is a discovery of the invention that, within the context of the brain environment, PDGF alone is sufficient to drive a robust proliferation of adult progenitor cells. The invention reveals a novel and unexpected relationship between brain tumors and neural progenitor cells. This relationship allows the amplification, and isolation, of large numbers of progenitors from the adult brain has not been achieved. Specifically, that the tumor creates a highly mitogenic environment that induces the massive proliferation of adult neural progenitor cells and that these progenitor cells retain the capacity to differentiate when removed from the mitogenic environment of the brain.

The in vivo proliferative environment is a highly organized (and complex) composition of many different cell types including progenitor cells, neurons, astrocytes, oligodendrocytes, and blood vessels. Each of these cellular components likely expresses multiple growth factors that are needed to create and maintain the proliferative environment. The methods of the invention can be used to more fully characterize the cellular and molecular details so the proliverative environment can be created in vitro. The invention provides that high levels of PDGF-B, expressed by a subset of progenitors, is sufficient to initiate the process in vivo. Examples of other growth factors and growth factor receptors which may be used within the context of the invention include other PDGFs, FGFs (e.g., FGF-2), neuregulins, including EGF and GGF (e.g., GGF-2), EGFR or any combination thereof.

Retroviral Vectors

To administer the growth factor or growth factor receptor to the brain, the methods of the invention utilize a retroviral vector. The retroviral vector comprises a nucleotide sequence encoding the growth factor and the vector is capable of expressing the growth factor. In one embodiment of the invention, the retrovirus is replication incompetent and therefore present little or no risk of spreading or of inducing a dangerous immune response. In another embodiment, the retrovirus is an amphoteric retrovirus, capable of infecting any cell type.

A retroviral vector of the invention can comprise a nucleotide sequence encoding a reporter molecule. In one embodiment, the growth factor is co-expressed with a reporter molecule. Co-expression can be achieved by an internal ribosome entry site (IRES). In one embodiment, the reporter molecule comprises a fluorescent protein. Non-limiting examples of fluorescent proteins include green fluorescent protein, yellow fluorescent protein, blue fluorescent protein and DsRed.

The retrovirus can further comprise regulatory sequences to make expression of the growth factor is inducible. Inducible expression can be achieved by using an inducible promoter. Non-limiting examples of inducible promoters are tetracycline inducible promoters (both “tet on” and “tet off” systems). With these systems, the expression of the growth factor could be turned on or off by administering doxyclycline to the animal. Another non-limiting example is to use an inducible cre recombinase system (such as cre-ER). With this approach, the retrovirally expressed growth factor is placed between two lox sequences which will allow the nucleotide sequence of the growth factor to be spliced and silenced upon treatment with tamoxiphen. The Cre system can be used to turn off (floxed) or turn on (stop-flox) expression of the growth factor or growth factor receptor. But, unlike the tet system, the cre induced changes in expression are irreversible.

The methods provided by the invention use retroviruses to deliver growth factors (PDGF-IRES-GFP and PDGF-IRES-DsRed) and growth factor receptors (EGFR-GFP) to glial progenitors in the adult rat brain. It is a discovery of the invention that adult glial progenitors possess an unexpected robust capacity to migrate, proliferate and form tumors that closely resemble human glioblastomas. As shown in the Examples, the invention provides methods for monitoring the migration and proliferation of the GFP+ and DsRed+ cells by time-lapse microscopy of slice cultures generated from retrovirus-infected brains. Slice cultures provide a convenient system to treat glial progenitors and glioma cells with growth factors and small molecule inhibitors (alone or in combination) and monitor the effects on migration and proliferation at the cellular level in the context of living brain tissue. Using flow cytometry and FACS for glial progenitor markers (A2B5 and O4), the Examples show that human gliomas contain an abundance of progenitor-like cells with tumorigenic potential. One can label these cells with GFP expressing retroviruses and transplant them into nude rat brain to determine the tumorigenic potential of specific subpopulations of cells. The methods of the invention can be used to define the key steps in growth factor signaling that drive adult glial progenitors and human glioma cells to form tumors and to use this knowledge to develop new strategies of targeted therapy.

There are a variety of animal models currently used to study gliomas. These include transplantation of rodent or human glioma cell lines. While these models are well characterized and very convenient (tumors form rapidly and consistently), the tumors tend to grow as well circumscribed pushing masses and do not resemble the heterogeneous and infiltrative character of human gliomas. This may be because the glioma-like characteristics have been altered or selected against in cell culture. More recently several transgenic mouse models that spontaneously form brain tumors have been developed (Weiss et al. 2002). These models have provided insight into the molecular defects that can give rise to gliomas. However, the time and location of tumor formation is variable, making them inconvenient to use for controlled treatment studies. A third approach has been to use retroviruses to deliver oncogenes to specific population of glial cells or progenitors in vivo by intracerebral injection. The advantage of this approach is that one can target a specific population of cells at a specific time and therefore know where and when the tumor initiating events occurred. This is particularly helpful in studying glioma cell dispersion since one can measure the distance of spread from the injection site. One of the best-characterized systems has been to use PDGF expressing retroviruses. The histologic type and grade of the tumors that form depends on the type of cells that are targeted and the genetic background of the animal (Dai et al. 2001) (Shih et al. 2004) (Hesselager and Holland 2003) (Hesselager et al. 2003) (Uhrbom et al. 2005).

The invention provides modifications to known retroviral systems and provides a novel glioma model with several advantages over previous glioma models. First, the invention targets adult glial progenitors. All previous models delivered the retroviruses to progenitor cells in the neonatal rodent brain. However, the most malignant gliomas occur in adults and therefore arise from cells that reside in the adult brain. There is evidence from cell culture studies that neonatal and adult progenitors differ in important ways—particularly with regards to their response to growth factors and in their capacity to migrate, proliferate and self-renew. For this reason, we feel it is essential to target and characterize adult progenitors.

A second advantage is that the invention utilizes amphoteric retroviruses that have proven to infect adult progenitors with high efficiency. As a result, the tumors form rapidly and consistently, 100% of the animals form tumors by 14 dpi. This is notably different from previous studies using other retroviral systems, in which tumors form in 30-50% of the animals and take months to form. These differences may be due to differences in the targeted cell type. Adult progenitors, targeted by the methods of the invention, may have a greater tumorigenic potential than neonatal progenitors. It is important to note that the amphoteric retroviruses we are using can infect a variety of species, including human cells. The PDGF retroviruses provided by the invention have also been used to generate tumors in several transgenic mouse lines. Thus, the methods of the invention can be used to test the effects on various genetic backgrounds without the need for crossing of mouse lines, as is required in previous retrovirus-based glioma models.

A third advantage provided by the invention is that the inventive retroviruses express high levels of GFP and DsRed, allowing identification of the infected cells (and their progeny) in brain tissue by fluorescence microscopy and isolation of the cells by FACS. Taking advantage of the fluorescent reporters, the invention provides methods to monitor the migration and proliferation of infected cells by time-lapse microscopy of slice cultures. The slice culture system allows one to deliver growth factor or small molecule inhibitors to the tumor tissue and monitor the effects on the behavior of individual cells. One can also monitor the cellular distribution of tagged proteins, such as EGFR-GFP.

Cell Replacement Therapy

Neural progenitor cells generated by the methods of the invention can be used for cell replacement therapy for the treatment of brain injury and disease, including multiple sclerosis, stroke, Alzheimer's disease and Parkinson's disease.

The invention provides methods for harnessing the highly proliferative environment of glial neoplasms to generate neural progenitor cells for cell replacement therapy in humans. To generate a model to study the behavior of human neural progenitor cells, progenitor cells are isolated from adult human white matter from surgical specimens and are infected in vitro with PDGF-expressing retrovirus and/or control retrovirus expressing only a fluorescent reporter gene. The infected human progenitors proliferate and express high levels of the reporter genes. The infected human progenitors can then be injected into nude rodent brains to determine if they will behave like the rodent progenitors. The human progenitors are then amplified, isolated and tested for their capacity to differentiate into mature brain cells that are useful for cell replacement therapy.

The invention also provides methods for regulating the rate of in vivo progenitor cell proliferation. For example, the retrovirus can be delivered directly into the brain of a human subject and replacement cells can be generated in vivo, without the need for removal of brain tissue or in vitro manipulation. One exemplary method for achieving regulation of cell proliferation is the use of inducible promoters to turn PDGF-B expression on and off.

Methods for administering cells to the brain and spinal cord are known in the art (e.g., U.S. Pat. Nos. 6,787,356, 6,444,205; 6,264,943, 5,762,926, 5,650,148; Surendran et al., Brain Res Dev Brain Res 153(1):19-27 (2004); Kondziolka et al., Cell Transplant 13(7-8):749-754 (2004); Oka et al., Brain Res 1030(1):94-102 (2004)).

Methods for administering retroviral vectors to the brain have been described in the art (see Consiglio et al., Proc Natl Acad Sci USA 101(41):14835-14840 (2004); Tanaka et al., Stroke 35(6):1454-1459 (2004)).

Animal Tumor Models, Therapy Development and Compound Screening Methods

The methods provided by the invention can be used to develop novel treatments for brain tumors. The methods of the invention will allow one to determine the autocrine and paracrine signaling mechanisms that drive glioma cells to migrate, proliferate and form tumors. The PDGF and EGFR models of the invention have permitted identification of a population of cells in the adult brain that possess a remarkable capacity to migrate proliferate and self-renew and if this proceeds unchecked it leads to the formation of tumors. Although the retroviral glioma models do not mimic the genetic complexity of human glioblastomas, they do mimic many of the histopathologic features of glioblastomas including an infiltrative growth pattern, marked vascular proliferation and palisading necrosis. The methods of the invention can be used to determine whether growth factor stimulated progenitors mimic the behavior of glioma cells and show a similar response to inhibitors of growth factor signaling. The retroviral models of the invention can be used to test the effects of a variety of compounds (and combinations of compounds), in slice culture and in vivo by, for example, convection enhanced delivery. The inventive methods and models can also be used in pre-clinical studies to develop a chemotherapeutic regime that can be brought to clinical trial.

The inventive methods can be used to elucidate the molecular mechanisms that regulate the proliferation, differentiation and survival of adult neural progenitor cells. PDGF-B expressing retrovirus is sufficient to initiate the generation of a highly proliferative environment in the adult brain, but the microenvironment created in this system is very complex and it is unlikely that PDGF-B is acting alone. The invention provides for other growth factors or signaling molecules that are present in the system and may account for the proliferative activity. In accord with the methods of the invention, a cocktail of two or more growth factors can be developed and delivered pharmacologically to regulate the proliferation, differentiation and survival of adult neural progenitor cells.

The methods of the invention also provide a system for studying the regulation of progenitor cell proliferation, differentiation and survival which can be used for development of new therapies for brain diseases, independent of cell replacement therapy. For example, in the treatment of malignant brain tumors, using the methods of the invention to identify the growth factors and other signaling molecules that drive the massive proliferation of progenitor cells may reveal new targets for chemotherapy.

Terms

In one aspect of the invention, the compound can be combined with a carrier. The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ≦20%.

The term “effective” is used herein to indicate that the inhibitor is administered in an amount and at an interval that results in the desired treatment or improvement in the disorder or condition being treated (e.g., an amount effective to modulate the growth of kidney tissue).

In some embodiments, the subject is a human, mouse, rabbit, monkey, rat, bovine, pig, sheep, goat or dog.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

The following examples illustrate the present invention, and are set forth to aid in the understanding of the invention, and should not be construed to limit in any way the scope of the invention as defined in the claims which follow thereafter.

### EXAMPLES

### Example 1

### PDGF Drives the Formation of Malignant Gliomas Via Autocrine and Paracrine Stimulation of Adult White Matter Progenitors

Progenitor cells in adult rat white matter were infected with a retrovirus that expresses high levels of PDGF. Tumors that closely resembled human glioblastomas formed in 100% of the animals by 14 days post-injection (dpi). The tumors were composed of a heterogeneous population of cells, the vast majority of which expressed markers of immature glia. Less than 20% of the tumor cells expressed detectable levels of the retrovirus reporter gene (GFP), suggesting that the majority of tumor cells were progenitors that had been recruited via paracrine growth factor signaling. A retrovirus expressing PDGF-IRES-DsRed was co-injected with a control retrovirus expressing GFP. The resulting tumors were composed of a mixture of red and green cells. Both populations were highly proliferative and infiltrative. Thus, adult white matter progenitors can be induced to behave like malignant glioma cells as a result of growth factor stimulation via autocrine and paracrine signaling.

Diffusely infiltrating gliomas are the most common type of adult brain tumor. Which cell type or types in the CNS give rise to gliomas is not clear. Gliomas and glial progenitors share a number of markers, such as, olig2, NG2 and PDGFRα ¹⁻⁵ suggesting that gliomas may arise from transformation of glial progenitors. This Example focuses on the large population of progenitor cells that resides in the adult white matter. Approximately 2% of the total cells in the adult rat white matter are cycling progenitors ^{6,7}. In humans the number may be as high as 4% ⁸. Adult white matter progenitors are a heterogeneous population ⁶, but the large majority appear to belong to the oligodendrocyte lineage ⁹⁻¹². Normally, adult glial progenitors are slowly cycling, and non-migratory, however, they can be induced to become more rapidly proliferative and migratory in response to growth factor stimulation ¹³⁻¹⁷.

PDGF is a well established mitogen for both neonatal and adult glial progenitors ^{13,15,18-21}. In fact, numbers of oligodendrocyte progenitors are in part controlled by PDGF levels ²²⁻²⁴. Many glial progenitors in the adult white matter express PDGFRα ^{12,16}, making them potential targets for both autocrine and paracrine PDGF signaling. Similarly, many human gliomas co-express PDGF and its receptors, suggesting that autocrine and paracrine signaling loops involving PDGF play an important role in glioma formation ^{25,26}. Delivery of PDGF expressing retrovirus into the neonatal mouse brain generates diffusely infiltrating gliomas that closely resemble human tumors ²⁷⁻²⁹. Furthermore, a retroviral construct that expressed higher levels of PDGF produced more malignant tumors with shorter latency and in a higher percentage of animals ²⁹. However, the possibility that PDGF may also drive tumor formation in the adult brain has not previously been tested.

This Example shows that infecting adult white matter progenitors with a PDGF-B-expressing retrovirus induces the formation of malignant gliomas with 100% efficiency. In addition, it is a discovery of the invention that the majority of the cells that comprise the tumors are uninfected glial progenitors that had been recruited and induced to proliferate via paracrine signaling.

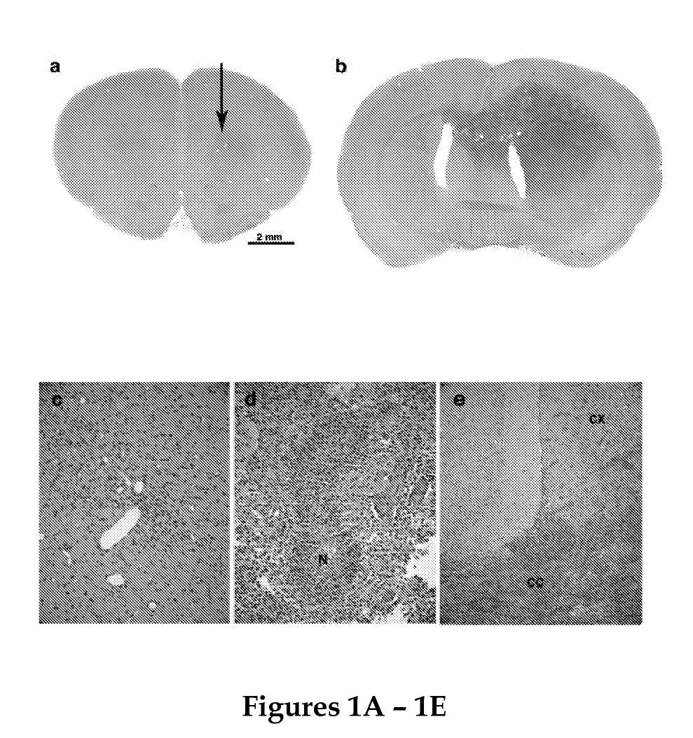

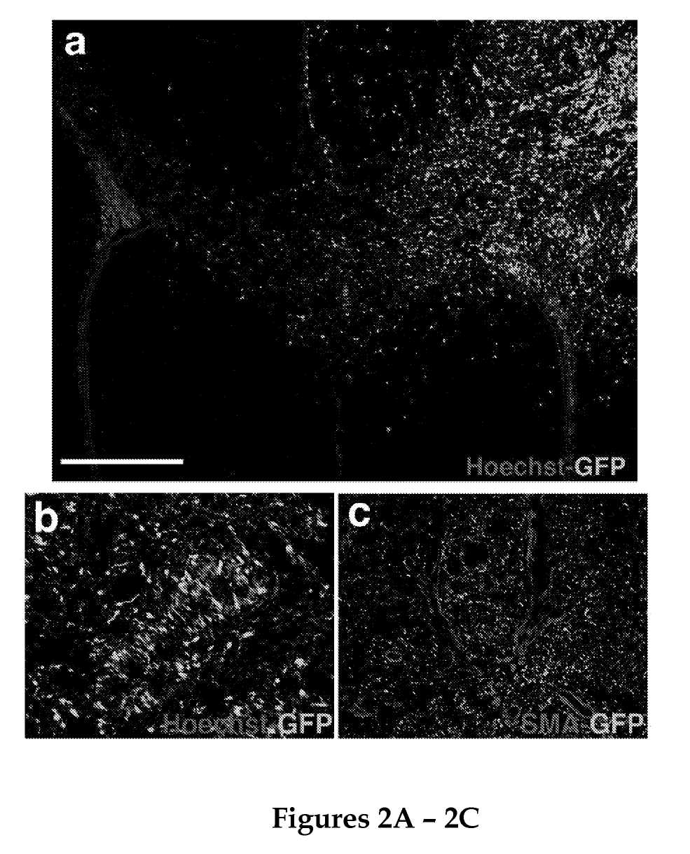

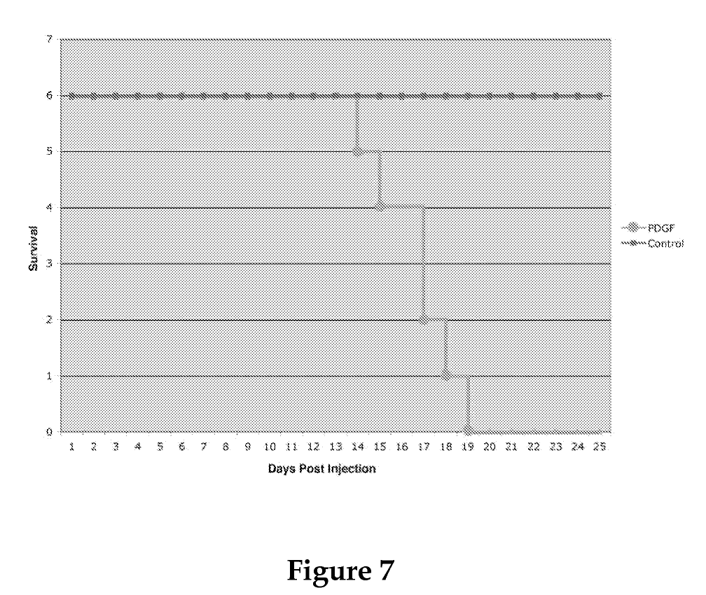

Infecting adult white matter progenitors with PDGF expressing retrovirus induces tumors that closely resemble human glioblastomas. To determine if PDGF overexpression could induce adult white matter progenitors to give rise to gliomas, a Moloney based retrovirus was generated that co-expresses PDGF-B-HA and GFP, separated by an internal ribosomal entry site (PDGF-IRES-GFP). Cells infected with the virus express the PDGF-B-HA construct at high levels (see below). The infected cells also express GFP at high levels, making it easy to identify the infected cells and their progeny in tissue sections by fluorescence microscopy and to isolate and analyze the cells by FACS. Five microliters of the retrovirus was stereotactically injected (at titers of approximately 10 ₅ cfu/ml) into the anterior subcortical white matter of adult rats (FIG. 1). Brain tumors developed in 100% of the animals injected (86 of 86) and a survival study showed that all animals developed signs of tumor induced morbidity between 14 and 19 days post injection (dpi) (FIG. 7). The tumors had all the histologic features of glioblastoma multiforme, including marked vascular proliferation as seen with immunostain for smooth muscle actin (SMA) (FIG. 2C) and pseudopalisading necrosis (FIGS. 1D and 2B). Fluorescence microscopy revealed that numerous GFP+ cells infiltrated the surrounding brain tissue and migrated across the corpus callosum into the contralateral hemisphere (FIGS. 1B, 1E, and 2A). The furthest infiltrating cells had migrated more than 5 mm from the injection site (FIGS. 1B and 2A). In contrast, none of the animals injected with control retrovirus (pQ-GFP) developed tumors (0 of 53) (FIGS. 1A and 1C).

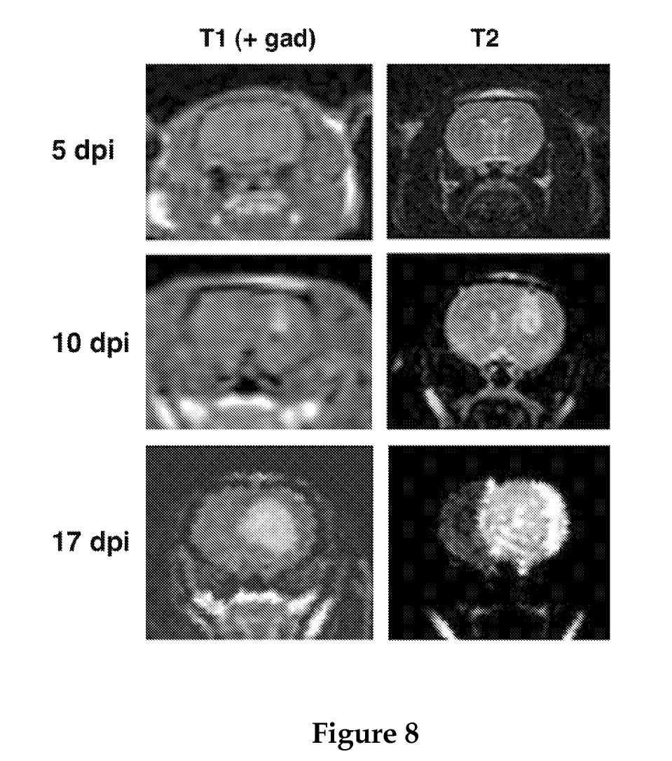

Tumor progression was also assessed through MRI scanning at 5, 10, and 15 dpi (FIG. 8). PDGF retrovirus induced tumors were visible by 10 dpi with some edema around the small tumor. By 15 dpi there was a large contrast enhancing tumor with marked edema throughout the affected hemisphere and extending into the contralateral hemisphere (FIG. 8)

To determine the degree of PDGF-B overexpression, ELISA was performed for PDGF-B levels on lysates generated from tumors (17 dpi with PDGF-IRES-GFP retrovirus) and control adult rat forebrain. The tumors showed approximately a 5000-fold increase in PDGF expression (24.2+/−4.5 ng/mg of protein) compared to control brains (4.3+/−2.1 pg/mg of protein).

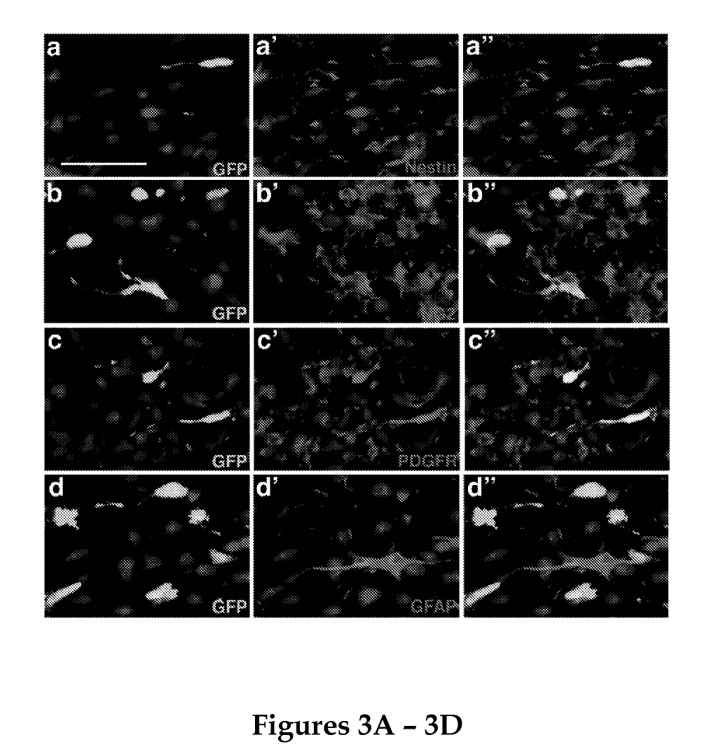

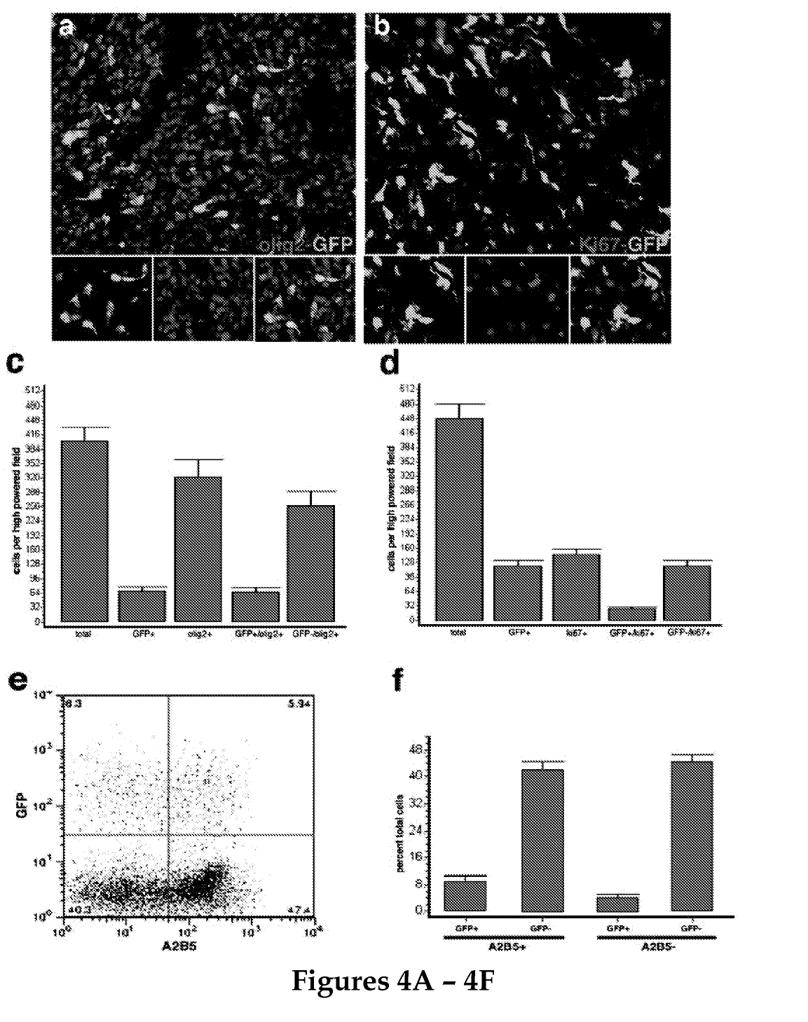

The tumors are composed of infected and uninfected glial progenitors. To characterize the cellular composition of the tumors, immunohistochemical analysis was performed for GFP and a variety of neuronal and glial markers. Only a subset of the cells in the tumor mass expressed detectable levels of GFP, showing that tumors were composed of both infected and uninfected cells (FIG. 3). The majority of tumor cells expressed markers seen in immature glia, including olig2, NG2, nestin, and PDGFRα and double immunofluorescence analysis showed that the vast majority of both GFP+ and GFP−cells were positive for these markers (FIGS. 3A-3C, and 4A). GFAP+/GFP−reactive astrocytes were scattered throughout the tumor and the surrounding brain tissue but less than 3% of the GFP+ cells were GFAP+ (FIG. 3D). None of the GFP+cells expressed the neuronal marker, NeuN.

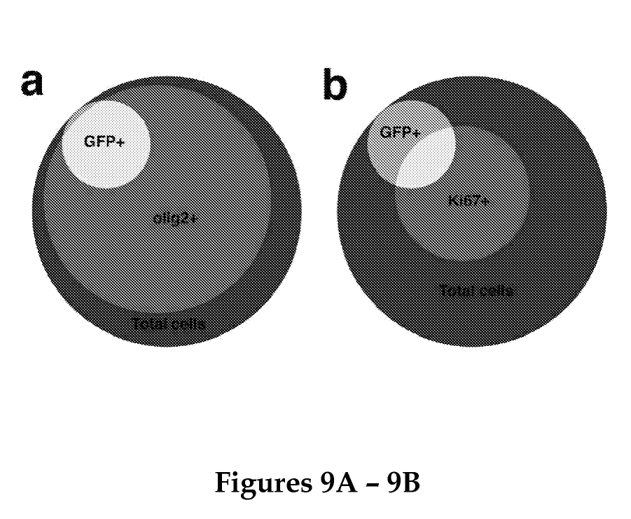

To determine the relative abundance and proliferation rates of GFP+ and GFP−cells in the tumors, quantitative analysis was performed for GFP and either olig2 or the proliferation marker Ki67 on 10 μm cryostat sections of tumors 17 dpi with the PDGF-IRES-GFP retrovirus. Total cells were counted using Hoechst nuclear stain. Only a minority (16.8+/−2.0%) of the tumor cells expressed detectable levels of GFP. The majority of tumor cells (77.6+/−4.6%) expressed olig2, regardless of GFP expression. The large majority of GFP+ cells (96.5+/−0.5%) were olig2+. Whereas, only a small percentage of the olig2+ cells (21.1+/−2.1%), were GFP+(FIGS. 4A, 4C, and 9). A large percentage of tumor cells (34.0+/−4.1%) were Ki67+. A similar percentage of GFP+ cells (39.1+/−3.3%) were Ki67+, but only a small percentage of the Ki67+ cells (18.2+/−1.8%) were GFP+ (FIGS. 4B, 4D, and 9). Thus, the majority of the tumor is composed of GFP−cells that have the phenotype of glial progenitors that had been recruited to proliferate via paracrine signaling.

To further quantitate the relative abundance of the different cell types in the tumor, flow cytometry was performed using the A2B5 monoclonal antibody, which is a well established marker of adult progenitor cells ^{9,16}. Flow cytometry showed that 13.2%+/−1.1% of the isolated cells expressed detectable levels of GFP (consistent with the immunohistochemical analysis). 51.2%+/−1.9% of the cells were A2B5+ but only 9.0%+/−2.6% were A2B5+/GFP+ (FIGS. 4E and 4F). From a typical experiment, approximately 5×10⁶ A2B5+ cells were isolated per tumor, of which the majority were GFP−. By contrast, using the same procedure, on the order of 10³-10⁴ A2B5+ cells are typically isolated from normal adult brain. Thus, the PDGF retrovirus caused a massive expansion of the A2B5+ population, most of which were GFP−(uninfected) cells.

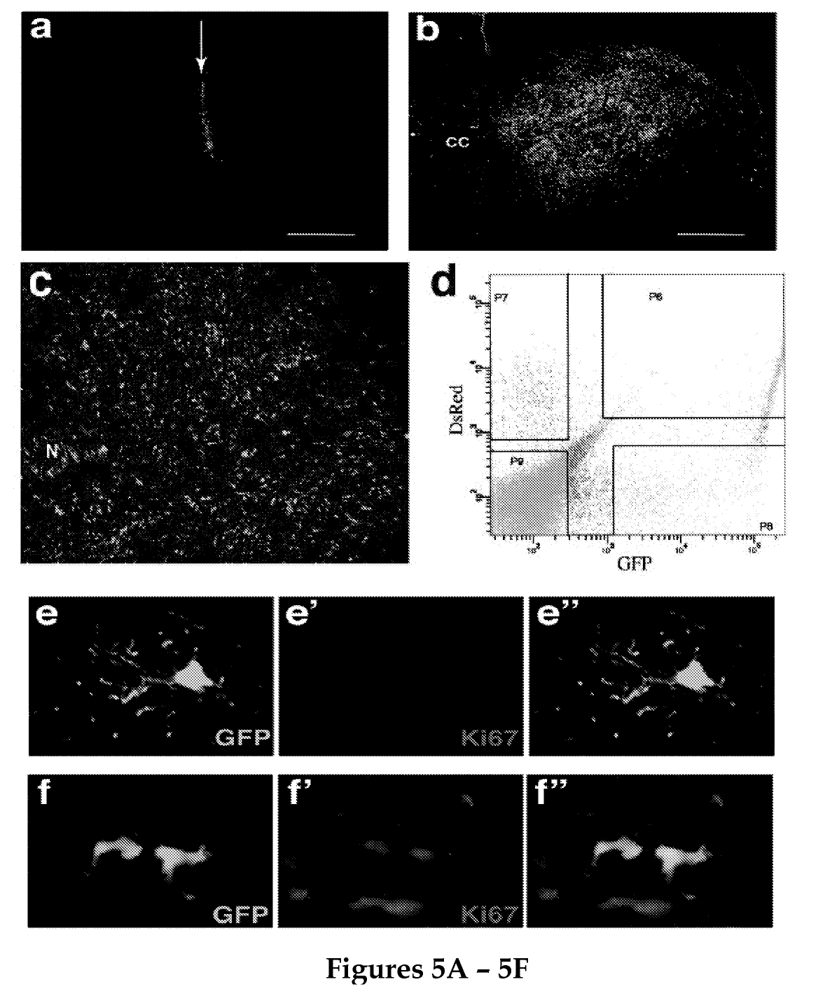

Adult white matter progenitors infected with the PDGF expressing retrovirus recruit and activate normal progenitors. To test the hypothesis that PDGF expressing cells were recruiting other glial progenitors to proliferate, pNIT-GFP and PDGF-IRES-DsRed retroviruses were co-injected into adult white matter. All of the rats co-injected with pNIT-GFP and PDGF-IRES-DsRed retroviruses formed malignant gliomas by 17 dpi (35 of 35) (FIGS. 5B and 5C). Immunofluorescence analysis and FACS for GFP and DsRed, showed that tumors were composed of 4 populations of cells: GFP−/DsRed−, GFP+/DsRed−, DsRed+/GFP−, and a small subpopulation was DsRed+/GFP+, demonstrating that coinfection was a rare event (FIG. 5D). The red and green cells were intermingled within the tumor and the large majority of both populations had an immature morphology (FIG. 5F) and were nestin+, NG2+, and olig2+. Both red and green cells diffusely infiltrated the brain, crossing the corpus callosum and invading the contralateral hemisphere (FIG. 5B).

As a control, the pNIT-GFP virus was injected alone (in the same volume and at the same titer, 10⁶ CFUs/ml, as used in the co-injections), and none of these animals developed tumors. By 17 dpi, the GFP cells remained few (approximately 100 cells per brain) and all of the GFP+ cells were distributed within 500 microns of the injection track (FIG. 5A). The majority of GFP+cells had acquired a complex morphology with numerous thin processes characteristic of differentiating oligodendrocytes (FIG. 5E). Immunofluorescence analysis showed that 77% of the GFP+ cells stained positive for the oligodendrocyte marker CC1 (61 of 79 cells counted), and none of the GFP+ cells expressed GFAP. Less than 5% of the GFP+ cells were ki67+ in brains injected with the control virus (FIG. 5E). In contrast, 26% of the GFP+ cells were ki67+ in tumors formed by the co-injection of the two viruses. Together, these results show that in normal adult white matter the majority of retrovirally infected cells left the cell cycle and differentiated along the oligodendrocyte lineage. However, within the mitogenic rich environment of the tumor, the pNIT-GFP infected cells were induced to proliferate and migrate, and thus mimic the behavior of malignant glioma cells.

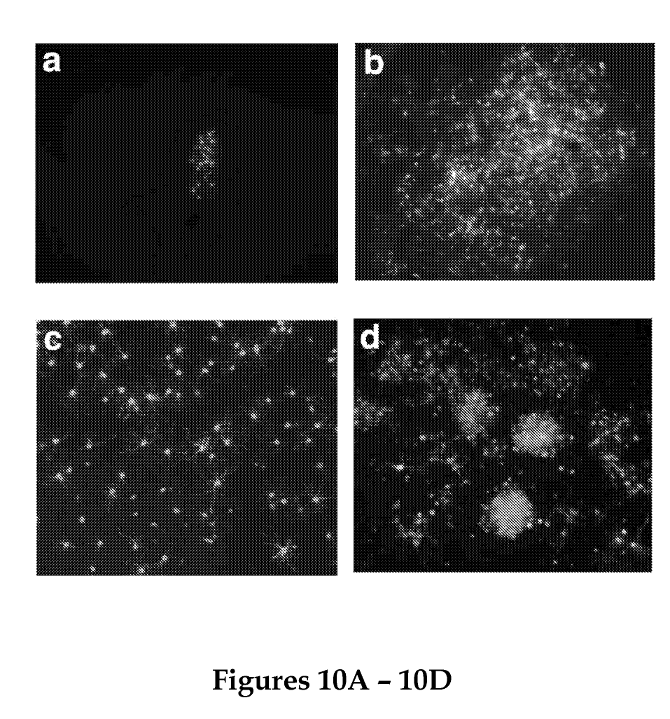

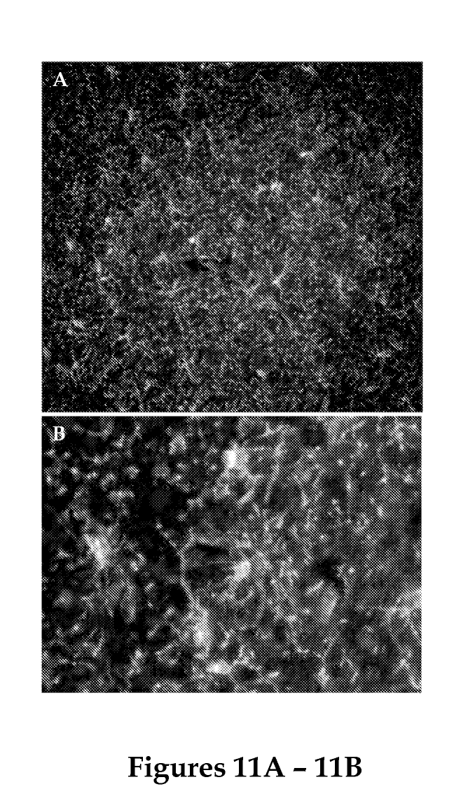

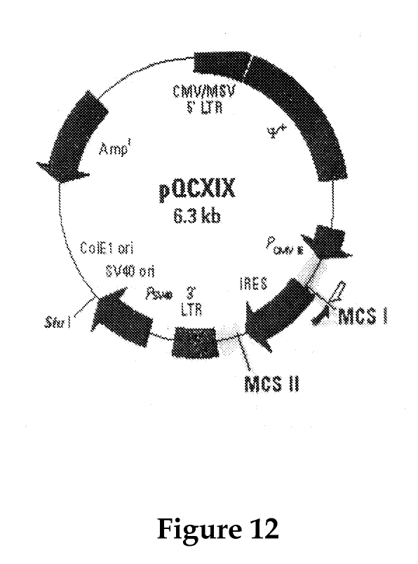

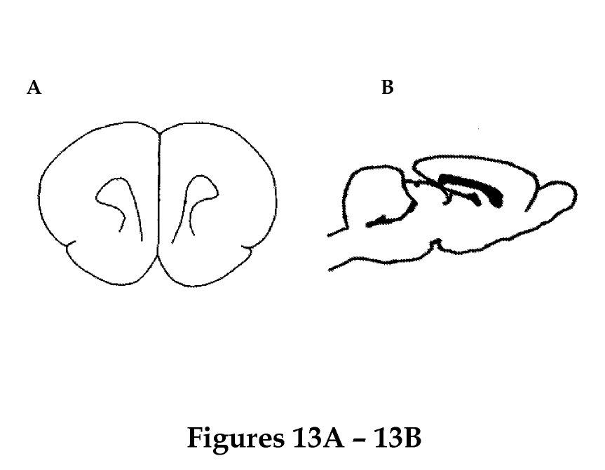

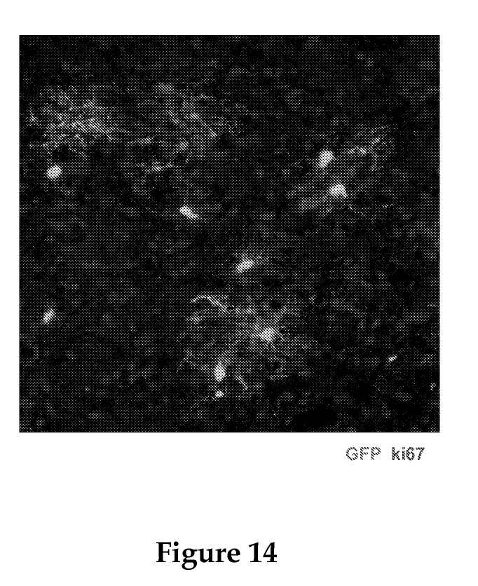

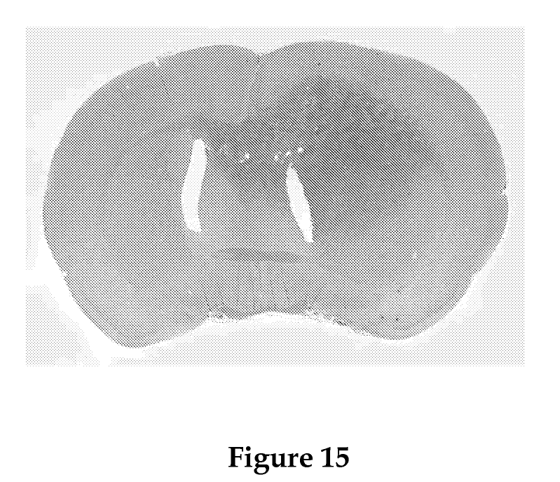

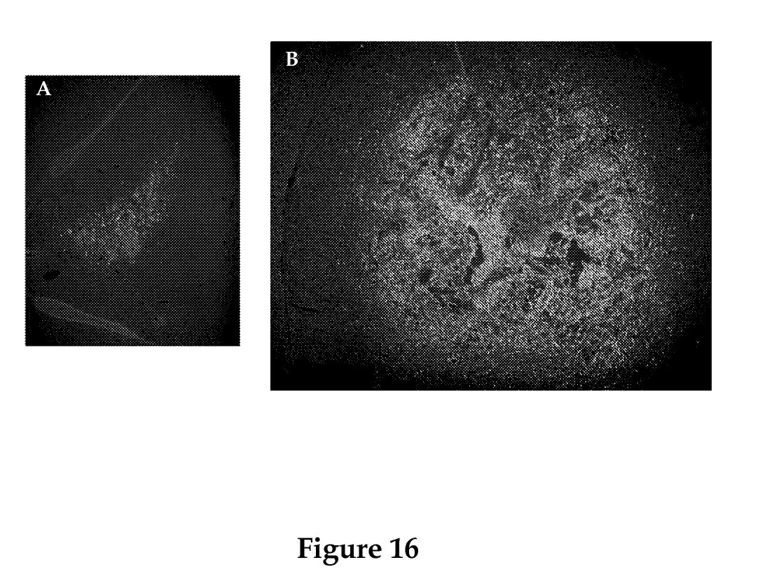

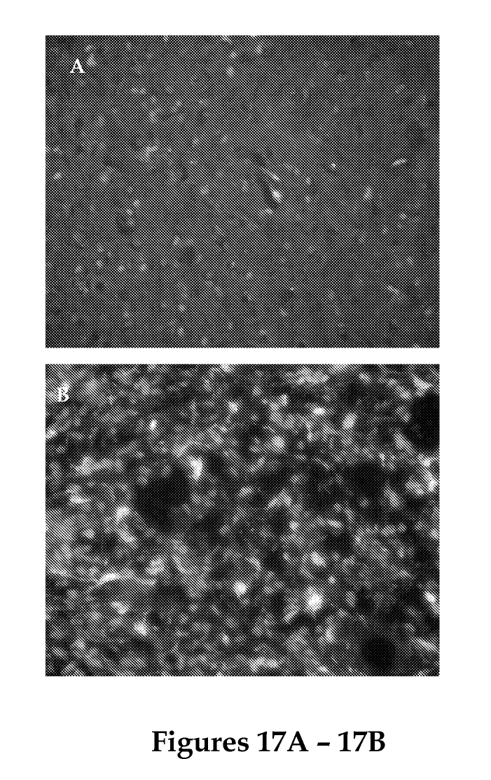

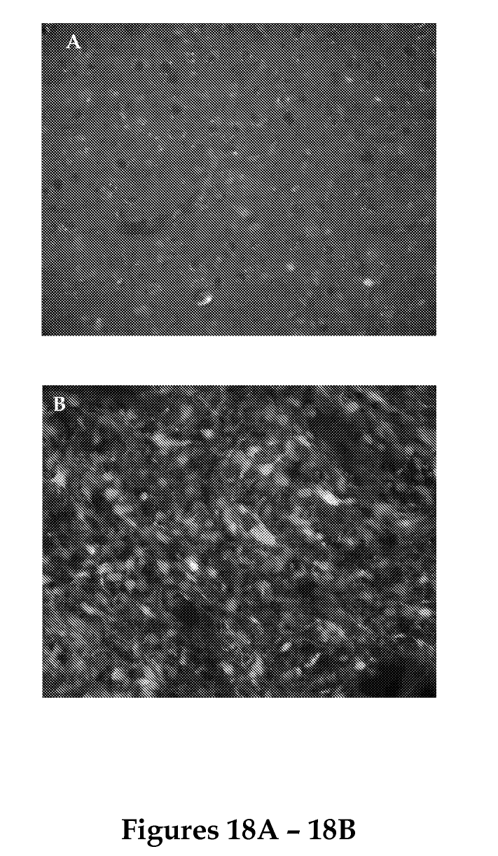

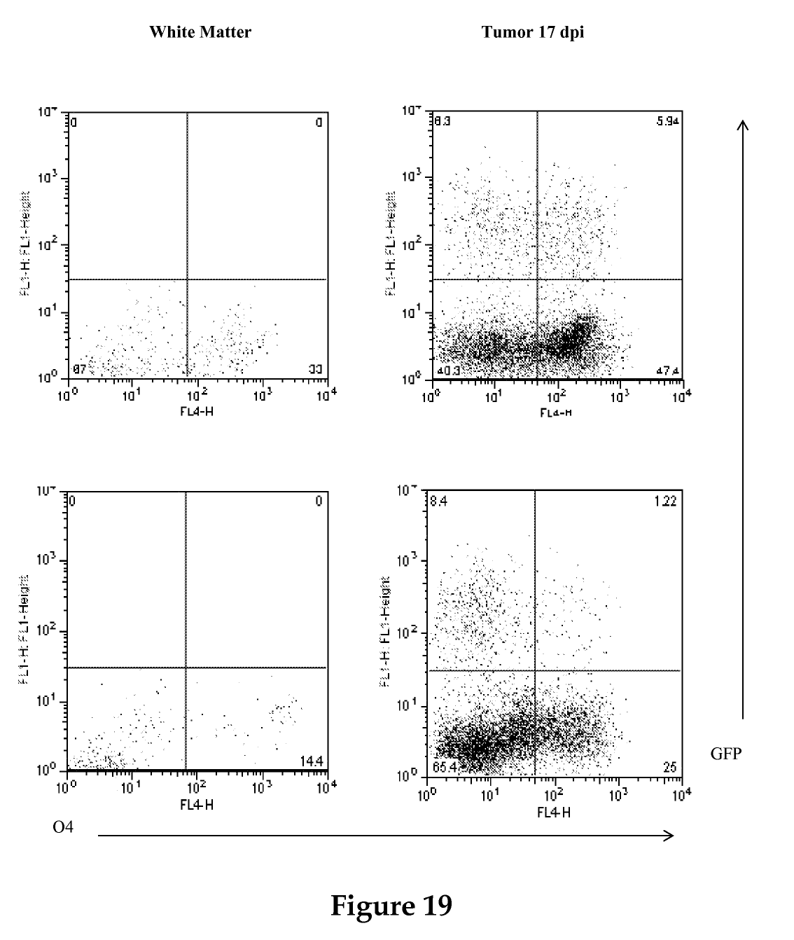

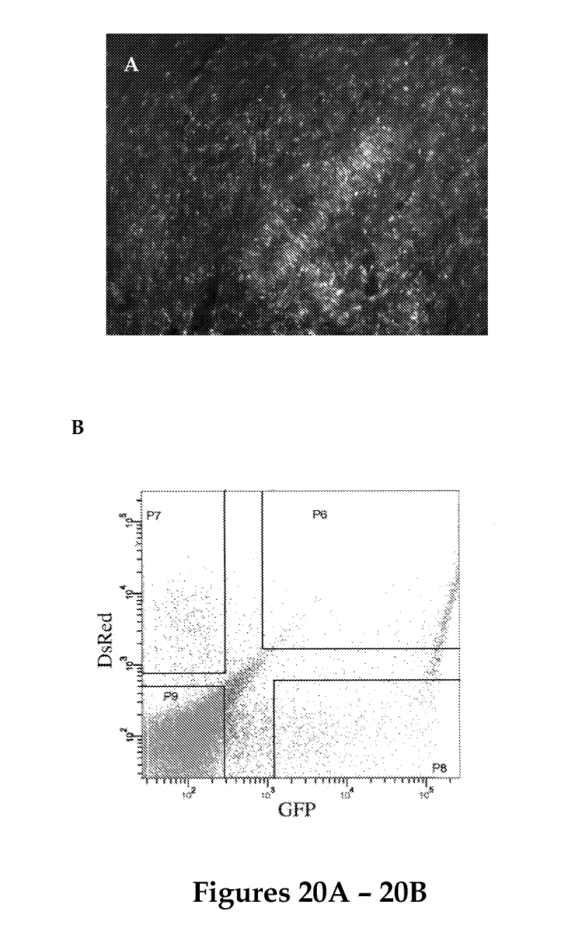

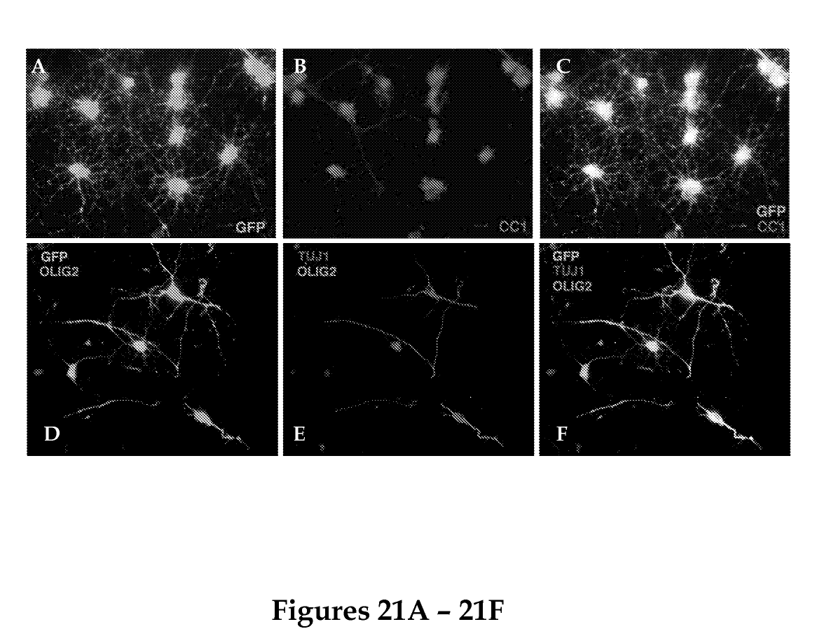

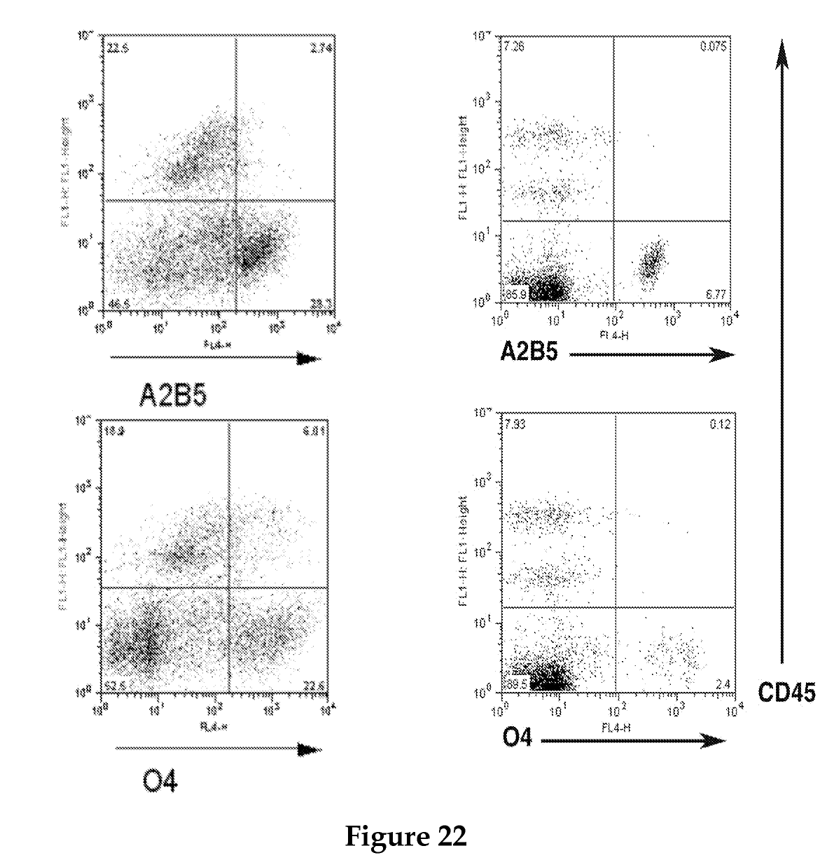

As a final test of the paracrine hypothesis, and to eliminate the possibility of any cells being co-infected with both viruses, progenitors were isolated from adult white matter, expanded in vitro for 5 days in B104 conditioned media and infected in vitro. Equal numbers of progenitors were separately infected with either pNIT-GFP or PDGF-IRES-DsRed. The cells were transplanted into adult rat subcortical white matter 2 days after infection, by either co-injecting pNIT-GFP+ cells and PDGF-IRES-dsRed+ cells (2,000 of each cell type), or GFP+cells alone (2,000). Co-injected cells formed infiltrative tumors by 20 dpi. The tumors were composed of three populations: DsRed+/GFP−, DsRed−/GFP+ and DsRed−/GFP−. No double positive cells were observed. Both the PDGF-IRES-dsRed and pNIT-GFP cells in the co-injections remained immature, infiltrative, and highly proliferative (FIG. 10). In contrast, pNIT-GFP cells injected alone remained few, acquired a branched morphology characteristic of differentiating glia, and did not develop into tumors (FIG. 10). All of the GFP+ cells were located within a few hundred microns of the injection site, demonstrating that when injected alone, the transplanted adult progenitors do not accumulate or migrate far from the injection site.

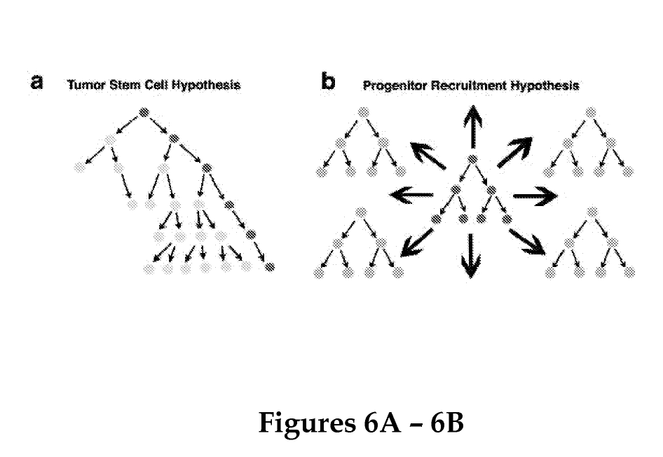

PDGF overexpression in adult white matter progenitors leads to glioma formation through paracrine and autocrine growth factor signaling. This Example shows that infecting glial progenitors in adult rat white matter with a retrovirus that expresses high levels of PDGF-B induces the rapid and consistent formation of tumors that closely resemble human glioblastomas. To explain how overexpression of a single growth factor can drive the formation of a malignant tumor so efficiently, the following model is proposed: the PDGF expressing retrovirus infects cycling adult progenitors, many of which express PDGFRαt. The infected cells secrete PDGF, which drives autocrine stimulation, resulting in expansion of the infected cell population and ever-increasing levels of PDGF secretion. The secreted PDGF also drives paracrine stimulation of the many uninfected PDGFRαt expressing progenitor cells near the injection site, causing expansion of the unlabeled population (FIG. 6B). Although over-expression of PDGF clearly initiates tumor formation in this model, other growth factors may be up-regulated (either by the progenitor cells or by other cells within the tumor) and cooperate with PDGF in the paracrine signaling.

Previous studies have shown that retroviral delivery of PDGF into neonatal mouse brains induces the formation of brain tumors. In these studies the frequency of tumor formation and the histologic features of the tumor (grade and tumor type) depended on the genetic background of the mice used, the population of progenitors targeted by the retrovirus, and the concentration of PDGF produced by the retrovirus ²⁷⁻³¹ One study suggested that malignant transformation of infected cells after incorporation of a PDGF-expressing retrovirus was a consequence of retroviral insertional mutagenesis of genes that cooperate with PDGF in gliomagenesis. Analysis of insertion sites showed several of the tagged loci harbored genes known to be involved in oncogenesis, indicating that additional molecular lesions played a role in the initiation of gliomagenesis in their experimental model ^{32,33}. The ability of PDGF to drive tumor formation also appears to be dose dependent. Using an avian retroviral system to deliver PDGF to neonatal progenitor cells that express the avian retroviral receptor off the nestin promotor, Shih et al. (2004) compared the tumorigenic effects of retroviruses expressing different levels of PDGF. The PDGF retrovirus expressing higher levels of growth factor caused more malignant tumors with shorter latency and in a higher percentage of animals. Furthermore, the tumors had marked vascular proliferation with recruitment of perivascular smooth muscle cells, suggesting that paracrine signaling was playing a role in tumor formation ²⁹.

The studies in this Example use the same high expressing PDGFB-HA construct cloned into an amphoteric retrovirus that also expresses high levels of a fluorescent reporter gene. This allows one to identify infected cells by fluorescence microscopy and flow cytometry. Results showed that PDGF-driven tumors that form in adult brain are largely composed of uninfected cells, showing that paracrine signaling is playing a major role in tumor formation. Furthermore, the rapidity with which gliomas developed and the 100% efficiency of glioma formation make it unlikely that retroviral insertional mutagenesis (and subsequent clonal expansion) played an essential role in initiating tumor formation. Rather, the tumors are formed by a polyclonal expansion of glial progenitors driven by autocrine and paracrine growth factor stimulation.

Heterogeneity of PDGF induced tumors and human malignant gliomas. Human gliomas are remarkably heterogeneous, both phenotypically and genetically ³⁴⁻³⁷. Cytogenetic analysis has shown that a single tumor may be composed of multiple subpopulations of tumor cells without any clear clonal relationship ^{38,39}. The clinical relevance of this heterogeneity is highlighted by studies showing that different subpopulations of gliomas cells show different sensitivity to radiation and chemotherapy ⁴⁰. One study has shown that cells isolated from human glioblastomas are also heterogeneous with regards to their capacity to initiate tumor formation ⁴¹. The tumor-initiating cells, which were isolated by FACS after surface labeling with an antibody against the stem cell marker CD133, comprised a minority of the tumor cells (19-29%) and were able to form new tumors when transplanted into the brains of nude mice whereas CD133− cells were not. The proposed model was that the CD133− cells represent the more differentiated progeny of the CD133+ cancer stem cells and have a limited capacity to self-renew (FIG. 6A). The results presented in this Example show another mechanism that may give rise to heterogeneous populations within gliomas. This model proposes that many genetically normal progenitor cells are recruited to the developing tumor and that their proliferation is driven in a growth factor dependent manner (FIG. 6B). The recruited cells may retain the capacity to differentiate if they are removed from the mitogenic environment of the tumor. Some of the recruited progenitors may also acquire new genetic lesions during their rapid proliferation, thus expanding the genetic heterogeneity of the tumors.
Glioma cells resemble glial progenitors. The malignant behavior of glioblastomas is generally assumed to require the accumulation of multiple genetic lesions. It is a discovery of the invention that over-expression of a single growth factor is sufficient to drive the formation of tumors with all the features of glioblastoma, including vascular proliferation, palisading necrosis, and diffuse infiltration. In the PDGF model adult glial progenitors have been driven to form these hallmark features of malignancy, suggesting that these features are the consequence of the inherent behavior of progenitor cells. For example, the tendency for glioblastomas to infiltrate across the corpus callosum closely resembles the normal tendency of progenitors cells to migrate through the subcortical white matter and across the corpus callosum during early postnatal development ⁴². Similarly, the tendency for glioma cells to accumulate around blood vessels and neurons may reflect an exaggerated form of the perivascular and perineuronal satellitosis that occurs in the normal brain.

Adult glial progenitors are normally less migratory and less proliferative than are neonatal glial progenitors, but become more migratory and proliferative in culture in the presence of growth factors PDGF and bFGF ¹⁵. The results in this Example show that when stimulated with high levels of PDGF, in vivo adult progenitors are induced to proliferate and migrate to a degree that more closely resembles the behavior of neonatal progenitors and that this behavior, in the context of ever-increasing PDGF levels, rapidly leads to the formation of malignant gliomas.

Despite their histologic and immunophenotypic similarities, the PDGF model provided by the invention does not harbor any of the genetic alterations that are commonly seen in human malignant gliomas and is thus an epigenetic model of malignancy that reveals the close relationship between adult glial progenitor and glioma cells.

### Methods

PDGF-GFP retrovirus construction and production. A 0.8 Kb fragment encoding PDGF-B-HA ²⁹ was ligated into the retroviral vectors pQ-MCS1-IRES-eGFP and pQ-MCS1-IRES-DsRed. Replication deficient viruses were generated by co-transfecting gp293 cells with a vsv-G plasmid and the control vector pQ-MCS1-IRES-eGFP (pQ-GFP), pQ-PDGFHA-IRES-eGFP (PDGF-IRES-GFP), or pQ-PDGFHA-IRES-DsRed (PDGF-IRES-DsRed) (Transfection kit, Invitrogen, Palo Alto, Calif.). Conditioned media containing viral particles was collected 24 hrs post transfection and centrifuged at 1,500 rpm, passed through a 0.45 μm filter, and centrifuged at 35,000 rpm for 1 hr at 4° C. to concentrate the virus. The pellet was resuspended in Opti-MEM (Gibco, Grand Island, N.Y.), aliquoted, and stored at −80° C. pNIT-GFP retrovirus was made from transfection of stably pNIT-GFP infected gp293 cells with vsv-G ⁴³. Virus collection and concentration was the same as above.
Animal Injections. Adult Sprague Dawley rats were anesthetized with 60 mg/kg ketamine and 6 mg/kg Xylazine (Henry Schein Pharmaceutical, Port Washington, N.Y.). Animals were placed in a stereotactic apparatus (Stoelting, Avondale, Ill.). Bregma was identified and a burr hole was made 2 mm lateral and 2.5 mm rostral. A 26 gauge Hamilton microsyringe (Reno, Nev.) was inserted to a depth of 3.5 mm and 5 μl of virus or 5 μl of cell suspension was injected at a rate of 0.2 μl/min. Brains were collected at 14 and 17 dpi with virus and 20 dpi with cells. Animals were anesthetized with ketamine-Xylazine prior to cardiac perfusion with 60 ml of PBS and 60 ml of 4% paraformaldehyde. Brains were post fixed for 48 hrs, then transferred to PBS until used. All animal experiments were performed according to the guidelines of the IACUC, Columbia University.
Staining Procedures. Fixed brains were cryosectioned at 10 μm. Antibodies used were: olig2; Nestin (rat 401); GFAP (DAKO, Carpinteria, Calif.); NeuN (Chemicon, Temecula, Calif.); GFP (Molecular Probes, Eugene, Oreg.); DsRed (BD Bioscience, San Jose, Calif.); Ki67 (NovoCastra, New Castle, UK); PDGFRα (NeoMarkers, Calif.), NG2; SMA (DAKO, Carpinteria, Calif.), CC1 (Oncogene, San Diego, Calif.), A2B5 (Hybridoma cell line, ATCC, Manassas, Va.). Sections were blocked with 10% NSG, 0.5% Triton X-100 for 30 min. prior to incubation overnight with primary antibody at 4° C. Sections were then washed and incubated in secondary antibody for 2 hrs. FITC, TRITC, and Cy5 (Jackson ImmunoResearch, West Grove, Pa.), conjugated secondary antibodies were used for double and triple fluorescence. Sections were stained with Hoechst 33342 (Molecular Probes, Eugene, Oreg.), for all nuclear stains. Hematoxylin and eosin stains were performed on 5 μm sections from paraffin embedded blocks.
Microscopy. Histologic sections were examined and photographed using a Zeiss axiophot 200 fluorescent microscope equipped with an axiocam (Zeiss) and openlab imaging software (Improvision). Micrographs were further processed using Adobe Photoshop.
Cell counting. Ten μm coronal cryosections of brains were examined at the level of the injection site at 17 dpi with the PDGF-IRES-GFP or pQ-GFP viruses. Adjacent sections were double-immunostained for olig2 and GFP or Ki67 and GFP and counterstained with Hoechst nuclear dye. Multiple 40× fields from 3 brains were photographed and the number of cells staining positive for each marker was manually counted using Adobe Photoshop. Statistical analysis was performed using InStat v3.0 program.
Cell culture. Adult white matter progenitor cell cultures were isolated from adult Sprague Dawley rats as previously described ^{6,16}. The isolated cells were resuspended in medium containing 30% B104 conditioned media and plated onto poly-1-lysine coated dishes. B104 conditioned medium was collected 48 hours after addition of basal media (DMEM, 0.5% FBS, PSA, T3, N2 supplement) to confluent cultures of this neuroblastoma cell line. After 6 days in culture, progenitor cells were infected with pNIT-GFP containing retrovirus or PDGF-IRES-DsRed containing retrovirus. Cells were injected into animals at a concentration of 2,000 cells/μl at 2 dpi. Some cells were maintained in basal media for 10 dpi to monitor the cells infected with pNIT-GFP vs. PDGF-IRES-DsRed (FIG. 10).
Flow Cytometry and Fluorescence Activated Cell Sorting. 17 dpi tumors were dissected and cells were isolated using the same method of isolation of adult white matter progenitors ^{6,16}. Tumor cells were stained in suspension with A2B5 antibody for 1 hr at 4° C. Cells were incubated for 30 min at 4° C. with an anti-IgM Cy5 secondary (Jackson ImmunoResearch, West Grove, Pa.). Cells were resuspended in PBS 10% FBS and FAC sorted in a BD FACSCaliber System (BD Bioscience, San Jose, Calif.). The FlowJo program was used for flow cytometry data analysis.
ELISA. PDGF-BB Quantikine Assay Kit (R&D Systems, Minneapolis, Minn.) was used to determine PDGF levels in whole forebrain lysates from 2 control and 2 PDGF retrovirus induced tumors (17 dpi). Ripa lysis buffer with protease inhibitors (Sigma, St. Louis, Mo.) was used for tissue lysis.

### REFERENCES

1. Ligon, K. L. et al. The oligodendroglial lineage marker OLIG2 is universally expressed in diffuse gliomas. J Neuropathol Exp Neurol 63, 499-509 (2004).
2. Chekenya, M. et al. The NG2 chondroitin sulfate proteoglycan: role in malignant progression of human brain tumours. Int J Dev Neurosci 17, 421-35 (1999).
3. Chekenya, M. & Pilkington, G. J. NG2 precursor cells in neoplasia: functional, histogenesis and therapeutic implications for malignant brain tumours. J Neurocytol 31, 507-21 (2002).
4. Bouvier, C. et al. Shared oligodendrocyte lineage gene expression in gliomas and oligodendrocyte progenitor cells. J Neurosurg 99, 344-50 (2003).
5. Shoshan, Y. et al. Expression of oligodendrocyte progenitor cell antigens by gliomas: implications for the histogenesis of brain tumors. Proc Natl Acad Sci USA 96, 10361-6 (1999).
6. Gensert, J. M. & Goldman, J. E. Heterogeneity of cycling glial progenitors in the adult mammalian cortex and white matter. J Neurobiol 48, 75-86 (2001).
7. Hommes, O. R. & Leblond, C. P. Mitotic division of neuroglia in the normal adult rat. J Comp Neurol 129, 269-78 (1967).
8. Roy, N. S. et al. Identification, isolation, and promoter-defined separation of mitotic oligodendrocyte progenitor cells from the adult human subcortical white matter. J Neurosci 19, 9986-95 (1999).
9. Nunes, M. C. et al. Identification and isolation of multipotential neural progenitor cells from the subcortical white matter of the adult human brain. Nat Med 9, 439-47 (2003).
10. Dawson, M. R., Polito, A., Levine, J. M. & Reynolds, R. NG2-expressing glial progenitor cells: an abundant and widespread population of cycling cells in the adult rat CNS. Mol Cell Neurosci 24, 476-88 (2003).
11. Levison, S. W., Young, G. M. & Goldman, J. E. Cycling cells in the adult rat neocortex preferentially generate oligodendroglia. J Neurosci Res 57, 435-46 (1999).
12. Nishiyama, A., Lin, X. H., Giese, N., Heldin, C. H. & Stallcup, W. B. Co-localization of NG2 proteoglycan and PDGF alpha-receptor on O2A progenitor cells in the developing rat brain. J Neurosci Res 43, 299-314 (1996).
13. Wolswijk, G., Riddle, P. N. & Noble, M. Platelet-derived growth factor is mitogenic for O-2Aadult progenitor cells. Glia 4, 495-503 (1991).
14. Shi, J., Marinovich, A. & Barres, B. A. Purification and characterization of adult oligodendrocyte precursor cells from the rat optic nerve. J Neurosci 18, 4627-36 (1998).
15. Wolswijk, G. & Noble, M. Cooperation between PDGF and FGF converts slowly dividing O-2Aadult progenitor cells to rapidly dividing cells with characteristics of O-2Aperinatal progenitor cells. J Cell Biol 118, 889-900 (1992).
16. Mason, J. L. & Goldman, J. E. A2B5+ and O4+ Cycling progenitors in the adult forebrain white matter respond differentially to PDGF-AA, FGF-2, and IGF-1. Mol Cell Neurosci 20, 30-42 (2002).
17. Ffrench-Constant, C. & Raff, M. C. Proliferating bipotential glial progenitor cells in adult rat optic nerve. Nature 319, 499-502 (1986).
18. Richardson, W. D., Pringle, N., Mosley, M. J., Westermark, B. & Dubois-Dalcq, M. A role for platelet-derived growth factor in normal gliogenesis in the central nervous system. Cell 53, 309-19 (1988).
19. Bogler, O., Wren, D., Barnett, S. C., Land, H. & Noble, M. Cooperation between two growth factors promotes extended self-renewal and inhibits differentiation of oligodendrocyte-type-2 astrocyte (O-2A) progenitor cells. Proc Natl Acad Sci USA 87, 6368-72 (1990).
20. Noble, M., Murray, K., Stroobant, P., Waterfield, M. D. & Riddle, P. Platelet-derived growth factor promotes division and motility and inhibits premature differentiation of the oligodendrocyte/type-2 astrocyte progenitor cell. Nature 333, 560-2 (1988).
21. Raff, M. C., Lillien, L. E., Richardson, W. D., Burne, J. F. & Noble, M. D. Platelet-derived growth factor from astrocytes drives the clock that times oligodendrocyte development in culture. Nature 333, 562-5 (1988).
22. Woodruff, R. H., Fruttiger, M., Richardson, W. D. & Franklin, R. J. Platelet-derived growth factor regulates oligodendrocyte progenitor numbers in adult CNS and their response following CNS demyelination. Mol Cell Neurosci 25, 252-62 (2004).
23. Calver, A. R. et al. Oligodendrocyte population dynamics and the role of PDGF in vivo. Neuron 20, 869-82 (1998).
24. van Heyningen, P., Calver, A. R. & Richardson, W. D. Control of progenitor cell number by mitogen supply and demand. Curr Biol 11, 232-41 (2001).
25. Hermanson, M. et al. Platelet-derived growth factor and its receptors in human glioma tissue: expression of messenger RNA and protein suggests the presence of autocrine and paracrine loops. Cancer Res 52, 3213-9 (1992).
26. Westermark, B., Heldin, C. H. & Nister, M. Platelet-derived growth factor in human glioma. Glia 15, 257-63 (1995).
27. Dai, C. et al. PDGF autocrine stimulation dedifferentiates cultured astrocytes and induces oligodendrogliomas and oligoastrocytomas from neural progenitors and astrocytes in vivo. Genes Dev 15, 1913-25 (2001).
28. Uhrbom, L., Hesselager, G., Nister, M. & Westermark, B. Induction of brain tumors in mice using a recombinant platelet-derived growth factor B-chain retrovirus. Cancer Res 58, 5275-9 (1998).
29. Shih, A. H. et al. Dose-dependent effects of platelet-derived growth factor-B on glial tumorigenesis. Cancer Res 64, 4783-9 (2004).
30. Hesselager, G., Uhrbom, L., Westermark, B. & Nister, M. Complementary effects of platelet-derived growth factor autocrine stimulation and p53 or Ink4a-Arf deletion in a mouse glioma model. Cancer Res 63, 4305-9 (2003).
31. Uhrbom, L., Hesselager, G., Ostman, A., Nister, M. & Westermark, B. Dependence of autocrine growth factor stimulation in platelet-derived growth factor-B-induced mouse brain tumor cells. Int J Cancer 85, 398-406 (2000).
32. Johansson, F. K. et al. Identification of candidate cancer-causing genes in mouse brain tumors by retroviral tagging. Proc Natl Acad Sci USA 101, 11334-7 (2004).
33. Johansson, F. K., Goransson, H. & Westermark, B. Expression analysis of genes involved in brain tumor progression driven by retroviral insertional mutagenesis in mice. Oncogene (2005).
34. Wikstrand, C. J., Grahmann, F. C., McComb, R. D. & Bigner, D. D. Antigenic heterogeneity of human anaplastic gliomas and glioma-derived cell lines defined by monoclonal antibodies. J Neuropathol Exp Neurol 44, 229-41 (1985).
35. Coons, S. W. & Johnson, P. C. Regional heterogeneity in the proliferative activity of human gliomas as measured by the Ki-67 labeling index. J Neuropathol Exp Neurol 52, 609-18 (1993).
36. Prados, M. D. & Levin, V., Biology and treatment of malignant glioma. Semin Oncol 27, 1-10 (2000).
37. Noble, M. & Dietrich, J. The complex identity of brain tumors: emerging concerns regarding origin, diversity and plasticity. Trends Neurosci 27, 148-54 (2004).
38. Coons, S. W. & Johnson, P. C. Regional heterogeneity in the DNA content of human gliomas. Cancer 72, 3052-60 (1993).
39. Coons, S. W., Johnson, P. C. & Shapiro, J. R. Cytogenetic and flow cytometry DNA analysis of regional heterogeneity in a low grade human glioma. Cancer Res 55, 1569-77 (1995).
40. Arbit, E., Shapiro, J. R., Fiola, M., Malkin, M. G. & Galicich, J. H. The significance of morphologically viable glioma cells found at the time of operation after interstitial brachytherapy. Neurosurgery 32, 105-10 (1993).
41. Singh, S. K. et al. Identification of human brain tumour initiating cells. Nature 432, 396-401 (2004).
42. Kakita, A., Zerlin, M., Takahashi, H. & Goldman, J. E. Some glial progenitors in the neonatal subventricular zone migrate through the corpus callosum to the contralateral cerebral hemisphere. J Comp Neurol 458, 381-8 (2003).
43. Kakita, A. & Goldman, J. E. Patterns and dynamics of SVZ cell migration in the postnatal forebrain: monitoring living progenitors in slice preparations. Neuron 23, 461-72 (1999).

### Example 2

### Methods for Generating Adult Neural Progenitor Cells

There are slowly cycling neural progenitors in the adult white matter (2% in rat and 4% in human). Their normal function is not known, but they may be used for cell replacement during repair of brain injury and disease. Previous studies have shown they can be isolated and amplified to a limited degree (up to 10,000 cell per brain). A method for generating more neural progenitor cells from adult brain represents a great advantage in the development of a cell replacement therapy.

Rationale for using a PDGF expressing retrovirus. PDGF is a powerful mitogen for progenitors during normal development. In vitro studies have shown that PDGF (in combination with other growth factors) is able to keep cells immature and cycling. PDGF receptors are expressed by adult progenitors. PDGF and its receptor are upregulated in gliomas. In vivo overexpression of PDGF via retrovirus has led to the formation of primary glial tumors in neonatal mice (Holland, Uhrbom).
Retrovirus is stereotactically injected into adult rat white matter. Adult coordinates from Bregma: 2 mm lateral, 3 mm rostral, and 4 mm ventral. Five microliters or one microliter of virus was injected at 0.2 microliters/minute. Animals were sacrificed at 2, 6, 10, 14 and 17 days post injection (dpi).

### Example 3

### PDGF-Driven Tumors

### Second Messenger Pathways, Role of EGFR, and Isolation of Subpopulations of Cells

Identifying the cells that give rise to glioblastomas has long been a major goal of neuro-oncology research. The underlying hypothesis of this line of research is that identifying the “cells of origin” will help to define the molecular mechanisms that control glioma migration, proliferation and survival and to develop strategies to target the pathways that drive tumor growth and spread. The recent discovery that some human gliomas contain a subset of cells with stem cell like properties has focused attention on neural stem cells of the adult subventricular zone (SVZ) as a likely “cell of origin” for many human glioblastomas (Singh et al. 2004a, Singh et al. 2003, Singh et al. 2004b) (Gil-Perotin et al. 2006) (Savarese et al. 2005) (Ignatova et al. 2002, Reya et al. 2001, Sanai et al. 2005). In addition to the SVZ, there is a very large population of glial progenitors that reside in adult white matter of both rodents and humans. It is estimated that these cells account for up to 4% of the total cells in the adult white matter, making them arguably the largest population of cycling cells in the adult brain (Dawson et al. 2000, Dawson et al. 2003, Gensert and Goldman 2001, Hommes and Leblond 1967, Roy et al. 1999). Normally, these cells are non-migratory and slowly cycling, however, they can be induced to migrate and proliferate rapidly when stimulated by growth factors (Shi et al. 1998) (Wren et al. 1992) (Wolswijk and Noble 1992) (Wolswijk et al. 1991). Adult glial progenitors are a heterogeneous population, but many of them express markers of the oligodendrocyte lineage including NG2, PDGFRα and olig2 (Dawson et al. 2000, Dawson et al. 2003, Gensert and Goldman 2001). Antibodies against surface gangliosides, A2B5 and O4, have proven to be very useful in isolating adult glial progenitors from dissociated tissue preparation (human and rodent) since the ganglioside antigens are not cleaved by protease treatment (Mason and Goldman 2002, Nunes et al. 2003, Roy et al. 1999). These antibodies have been used to isolate subpopulations of progenitor-like cells from human gliomas. These findings are consistent with previous results showing that human gliomas express markers also expressed by adult glial progenitors, including olig2, NG2 and PDGFRα, suggesting a close relationship (Bouvier et al. 2003, Chekenya and Pilkington 2002, Ligon et al. 2004, Shoshan et al. 1999).

### The Cancer Stem Cell Hypothesis Vs. the Progenitor Cell Hypothesis

The “cancer stem cell hypothesis” states that a (small) subset of the tumor cells have stem-cell like features and that these are the cells that give rise to tumors and to tumor recurrences while the bulk of tumor cells are “partially differentiated progeny” that have a limited capacity to self-renew and thus can not give rise to new tumor growth. This hypothesis points to a specific subpopulation of cells to target. One underlying assumption of the cancer stem cell hypothesis is that many or most tumor cells irreversibly differentiate to a point where they no longer have the capacity to form new tumors. An alternative hypothesis, the “progenitor cell hypothesis,” states: 1) gliomas arise from glial progenitors and are predominantly composed of cells that closely resemble glial progenitors, 2) the migration and proliferation of these cells is regulated by growth factor stimulation from the environment, and 3) these cells do not terminally differentiate, but rather retain the capacity to migrate, proliferate and self-renew indefinitely. If glioma cells can move back and forth between different states of differentiation, then one cannot hope to use specific marker to identify and target a subpopulation of “cancer stem cells.”

The progenitor hypothesis points to a different therapeutic strategy; that is to target the growth factor signaling mechanisms that drive progenitor cell proliferation and migration. The value of isolating and studying specific subpopulations of glioma cells, as provided by the invention, will be to characterize the mechanisms that drive their migration and proliferation and develop strategies to block it. For this information to be relevant, one must study the right types of cells in the right environment. Human glioblastomas are notoriously heterogeneous, both genetically and phenotypically, and it is very unlikely that there is one “right cell” to study. It is possible that some gliomas arise from astrocytes or neural stem cells and others arise from glial progenitors. Still, the majority of gliomas contain an abundance of cells with the morphology and immunophenotype that resemble glial progenitors. It is a discovery of the invention that it is the proliferative and migratory capacity of these cells that accounts for the aggressive growth of human gliomas.

### Growth Factor Signaling in Glial Progenitors and Gliomas

Several growth factors have been implicated in regulating gliogenesis during normal brain development. Among these, PDGF and EGF are well-characterized and established to play an role in the regulation of progenitor cell migration proliferation, differentiation and survival. These two growth factors have also been implicated in the pathogenesis of human gliomas. The EGFR gene is amplified in approximately 40% of primary glioblastomas and EGFR is overexpressed in over 60% of glioblastomas. Similarly, PDGF ligands and receptors are overexpressed in many human gliomas, suggesting the possibility of both autocrine and paracrine PDGF signaling loops (Westermark et al. 1995) (Hermanson et al. 1992). Several experimental systems have confirmed that both PDGF and EGF have powerful effects on glioma cell migration, proliferation and survival (Frappaz et al. 1988, Lammering et al. 2001) (Brockmann et al. 2003, Holland et al. 1998, Shih and Holland 2005). PDGF and EGF signaling affects adult glial progenitors in very similar ways; both drive glial progenitors to migrate and proliferate and (eventually) to form tumors that closely resemble human gliomas.

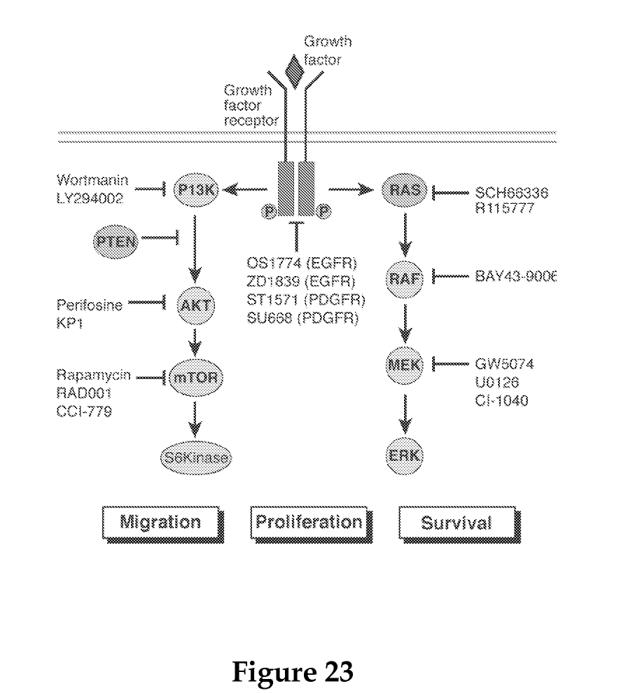

Both PDGFRs and EGFR belong to the larger family of receptor tyrosine kinases. The signaling cascades that are activated by these receptors have been extensively studied in a number of systems. The details vary depending on the cell type studied, but some general principles have emerged. 1) The binding of ligands to the extracellular domains induces receptors to dimerize and trans-phosphorylate tyrosine residues on their cytoplasmic domains. This leads to the activation of several second messenger signaling pathways, including (among others) the well-characterized RAS-MAP and PI3K-AKT signaling pathways (FIG. 23). These signaling cascades involve the reversible phosphorylation events that regulate the associations and enzymatic activity of the various signaling molecules. The development of phospho-specific antibodies has made it possible to characterize the activation of these pathways in situ and at the cellular level (Mandell 2003).

Both PDGFR and EGFR can activate many of the same pathways, creating the potential for cross-talk. PDGFR and EGFR can form functioning heterodimers (Saito et al. 2001) although the biological relevance of this type of interaction is not well established (Graves et al. 2002). Evidence from molecular analysis of human tumors suggests that PDGFR and EGFR are genetically amplified in separate subsets of gliomas, suggesting that gliomas may preferentially utilize one or the other receptors. However, amplification and/or overexpression of both EGFR and PDGFR are sometimes seen in the same tumor (Joensuu et al. 2005). Similarly, during normal brain development there is a transition from EGF responsive progenitors to predominantly PDGF responsive progenitors, although, there is a stage in glial progenitor development during which both receptors are expressed (Gago et al. 2003). This stage likely corresponds to the highly migratory NG2+ progenitors seen in the postnatal SVZ and subcortical white matter (Aguirre et al. 2005). This Example shows that glial progenitors that are infected with the EGFR-GFP expressing retrovirus continue to express PDGFRα for at least 15 weeks post infection and these cells seem to be “stuck” in a highly migratory and proliferative state. The glioma cells may be similarly “stuck” in such a state, and that inhibiting growth factor signaling will release them from the pathological drive to migrate and proliferate.

There are numerous clinical trials underway that are investigating the efficacy of various inhibitors that target growth factor signaling (Rich and Bigner 2004). However, the success of this approach has been limited. This is likely due to the complexity and redundancy of the signaling pathways. It is becoming evident that a combinatorial approach will be needed. Testing new combinations of drugs on cells in culture is informative, but may not predict the response in vivo. The need for convenient and realistic animal models is clear. The invention provides such a model.

### Adult Glial Progenitors are Driven to Form Tumors by PDGF Expressing Retrovirus

Example 1 shows the effect of PDGF expressing retroviruses on progenitor cells in the adult rat white matter (See also Assanah et al., 2006). This Example describes the analysis of glioma cell migration and proliferation of transplanted C6 glioma cells using time-lapse microscopy of brain slice cultures (Farin et al., 2006) and other experimental systems described below.

### Phospho-Specific Antibodies Reveal Activation of Down-Stream Signaling Pathways

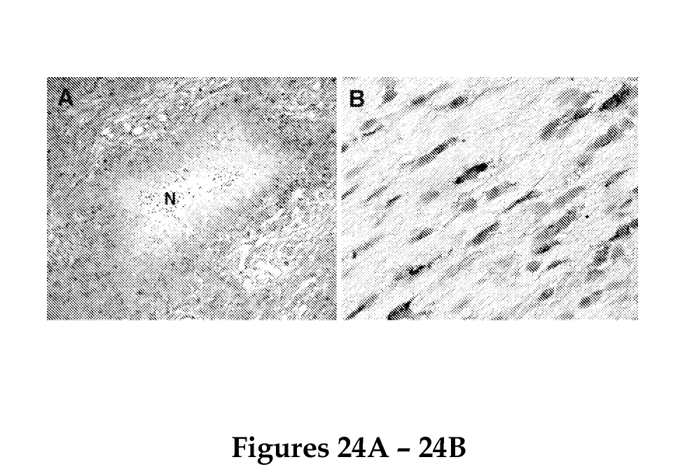

To characterize second messenger pathways activated in the PDGF driven tumors, immunohistochemical analyses have been performed using phospho-specific antibodies, starting with signaling along the P13 kinase pathway. The immunohistochemical stains for phospho-S6Kinase, a downstream target of mTOR. PDGF driven tumors stain strongly positive with antibodies against phospho-S6K (FIG. 24). Very high levels are seen in a subset of progenitors and glioma cells infiltrating the white matter and in subpial accumulation of cells. Little or no staining is seen in areas of pseudopalisading necrosis. These results show that the level of phospho-S6K is dependent on local environment.

### Time-Lapse Analysis of Slice Cultures Generated from the PDGF Driven Tumors

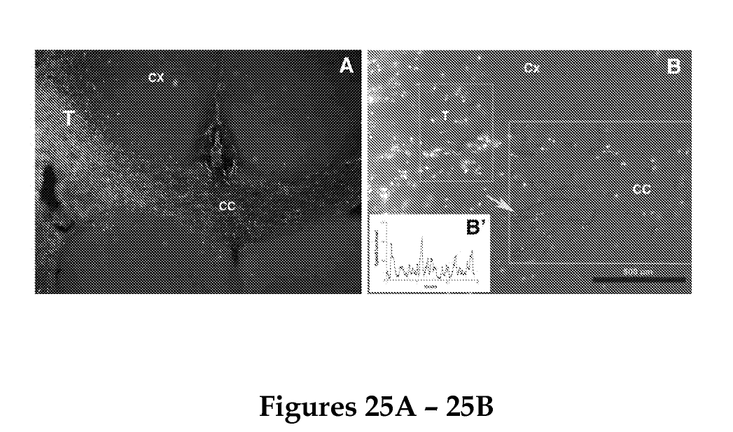

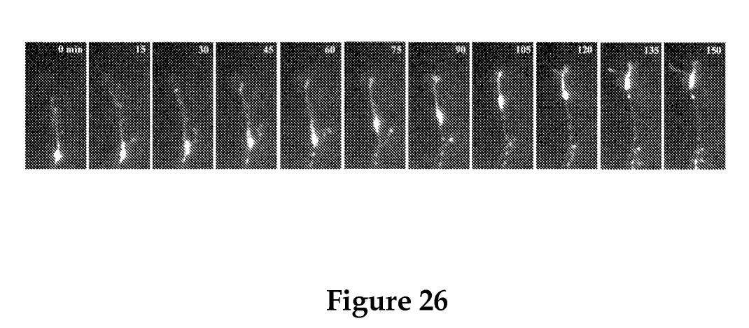

To characterize the migratory and proliferative behavior that leads to tumor formation after PDGF retrovirus, time-lapse microscopy was performed on slices generated from brains at 10 dpi with the PDGF expressing retrovirus into neonatal rat brains. At this time point 100% of the animals have formed a tumor at the injection site with cells infiltrating the surrounding brain both along blood vessels and through white matter tracts. Many cells can be seen crossing the corpus callosum into the contralateral hemisphere (FIG. 25). Time-lapse microscopy has revealed that the cells migrate in a saltatory fashion that closely resembles that seen by normal glial progenitors (Kakita and Goldman 1999) (Suzuki and Goldman 2003) and transplanted rat glioma cells (Farin et al. 2006). The cells have a long leading process that extends in the direction of migration (FIG. 26). Migrating cells frequently stop and divide en route, usually stopping for approximately 1 hour before the daughter cells resume migrating. Proliferation rate (% of total GFP+ cells that divide per hour) can be directly determined. The paths of migration were tracked using the DIAS image analysis (as described in Farin et al., 2006). The DIAS program can measure several parameters of cell movement, including speed, distance and directionality of migration. This analysis allows for quantitative measures of both migration and proliferation and serves as a basis with which to compare the effects of the various inhibitory compounds. The analysis shows that cells crossing the corpus callosum migrate faster and with greater directionality than do cells migrating within the main tumor, which stop and change direction with greater frequency (FIG. 25). These studies show the effects of growth factor stimulation on progenitor cell migration and proliferating in living brain tissue. These types of experiments have been performed on tumors formed by injecting the PDGF retrovirus in either neonatal or adult brains. The studies that are described below were performed on adult rats.

### EGFR-GFP Keeps Glial Progenitors Immature, Migratory and Proliferative

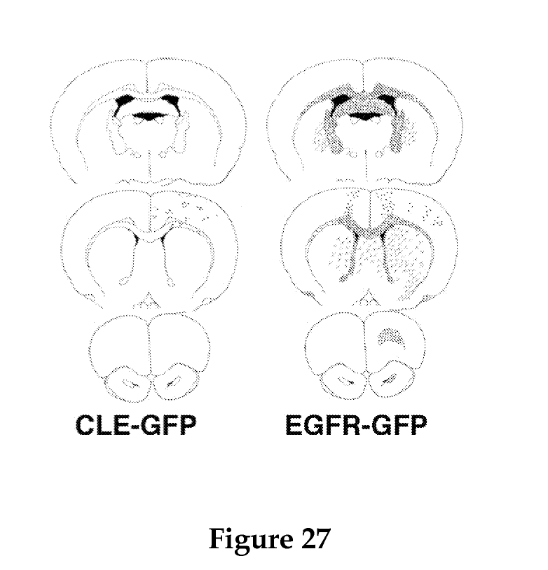

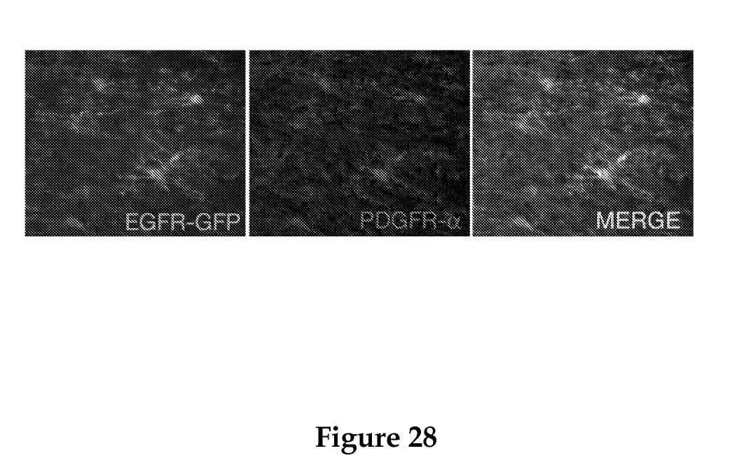

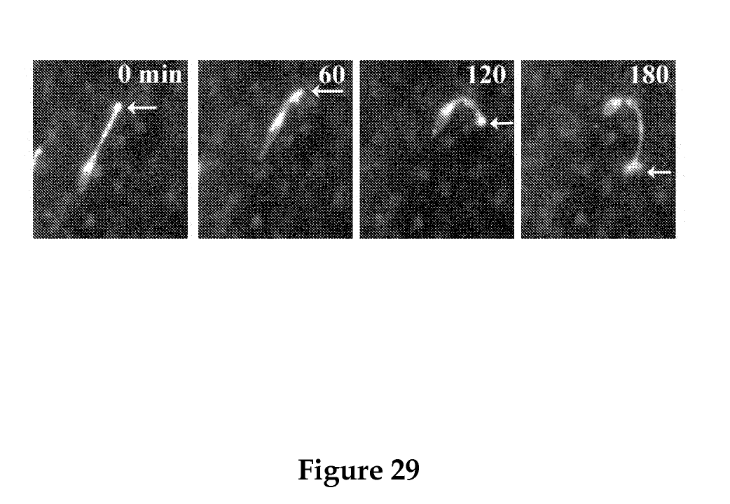

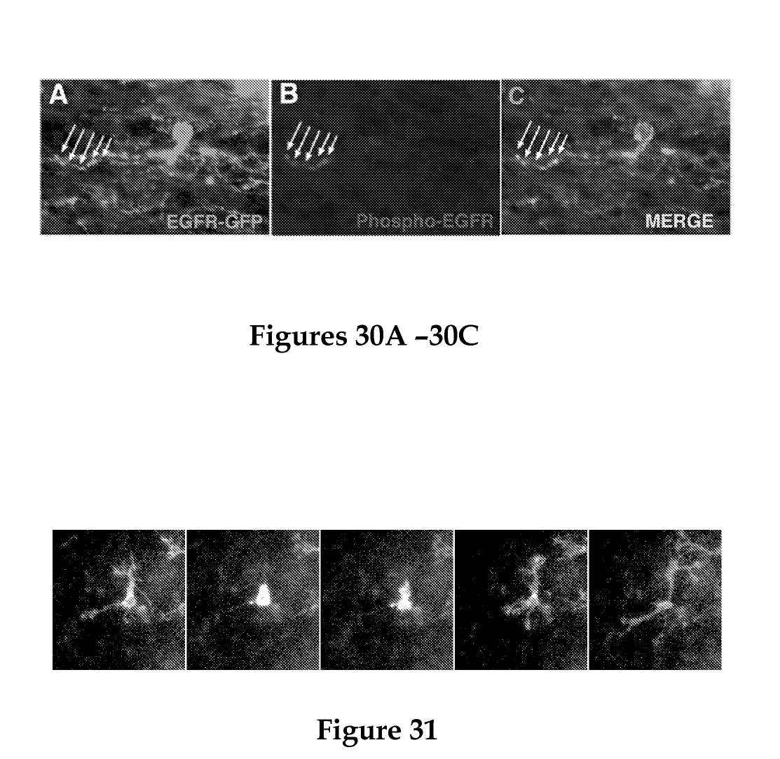

EGFR amplification is the most common genetic lesion in human glioblastomas. Numerous studies have implicated EGFR signaling in various aspects of glioma cell behavior including migration, proliferation and survival. It is also been shown to play role in progenitor cell migration and proliferation during embryonic and early postnatal development (Aguirre et al. 2005). To test the effects of constitutive expression on glial progenitors migration, proliferation and differentiation, glial progenitors were infected in the neonatal white matter with a retrovirus (10⁵ CFU in 1 μl) that expresses EGFR-GFP fusion protein or a control virus (CLE-GFP). Animals were sacrificed at several time points from 5 dpi to 15 wpi and the distribution, proliferation index and immunophenotype of EGFR-GFP+ cells was characterized by immunofluorescence analysis. At all time points analyzed the EGFR-GFP+ cells remained proliferative (KI67+) and immature (NG2+/PDGFRα+/Olig2+/GFAP−) (FIG. 28). Over the course of 15 weeks the distribution of the EGFR-GFP cells continued to extend away from the injection site as the cells infiltrated the surrounding brain tissue, predominantly along white matter tracks (FIG. 27). As a result of the continuous migration and proliferation the rats developed a diffuse hypercellularity of the white matter (reminiscent of gliomatosis cerebri). In contrast, cells infected with the control GFP virus did not expand in number and remained relatively close to the injection site (FIG. 27). Time-lapse analysis was also performed on the EGFR-GFP+ cells migrating in slice cultures generated at 5 dpi. This analysis showed that the EGFR-GFP cells are highly migratory and proliferative (FIGS. 29 and 31). One advantage to the EGFR-GFP model is that the GFP signal allows us to monitor the subcellular distribution of the EGFR-GFP as the cells migrate through the living brain tissue. EGFR-GFP accumulates in the leading process, suggesting a role in directed migration. Immunofluorescence analysis for phospho-EGFR shows punctate staining distributed along the leading processes of some cells which co-localizes with the distribution of EGFR-GFP (FIG. 30), showing that a localized fraction of the EGFR-GFP fusion protein is actively engaged in signaling.

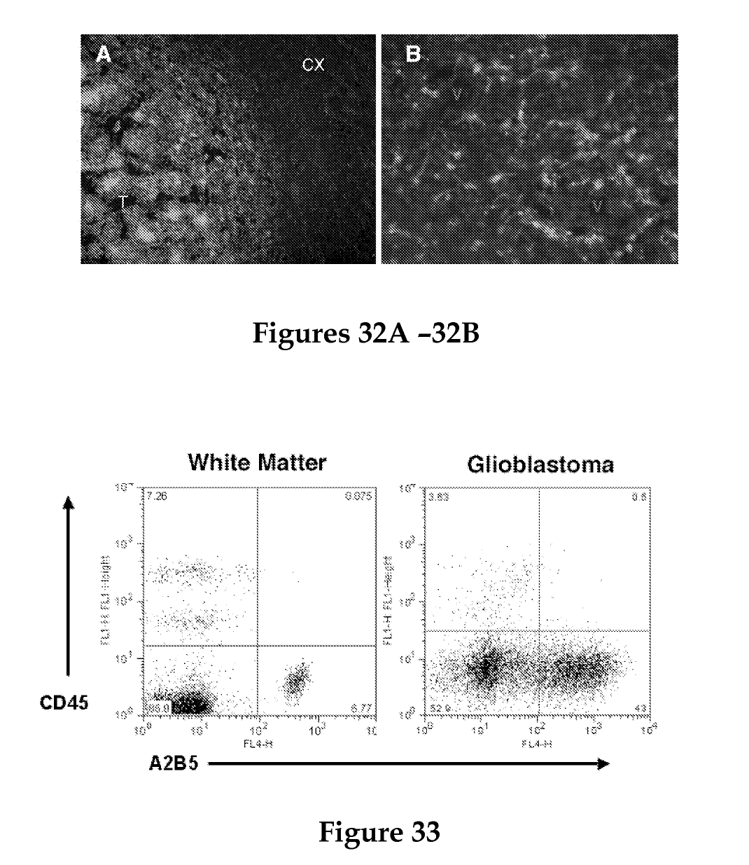

To determine whether EGFR expression is keeping the cells migratory and proliferative, long term survival studies have been performed on neonate and adult rats that have been injected with the EGFR-GFP fusion protein (12 rats) or control virus (6 rats). These studies are ongoing and most of the animals have not yet developed signs of tumor-induced morbidity. However, 3 of the animals have developed large malignant tumors (at 6 months to 1 year post injection). The tumors are composed predominantly of GFP+ cells, showing that the tumors arise from retrovirally infected cells and are a direct consequence of EGFR-GFP expression. None of the animals injected with control virus have formed tumors (FIG. 32).

### Human Gliomas Contain Cells that Resemble Glial Progenitors

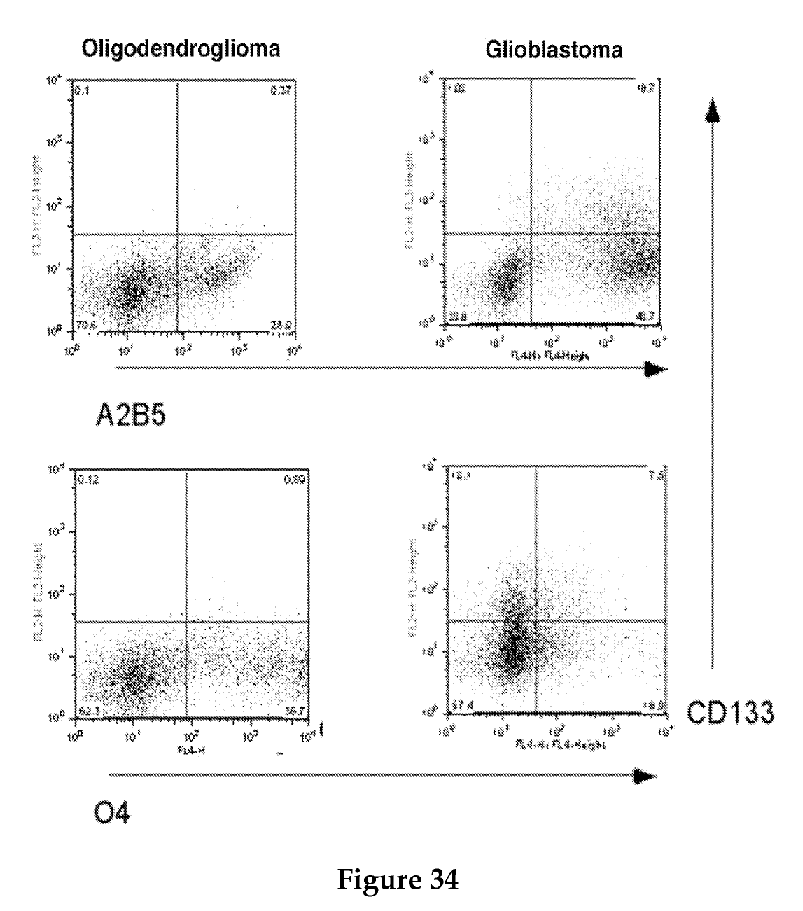

Flow cytometry and FACS have been used to characterize and isolate subpopulations of cells from human glioma specimens on the basis of stem cell markers (CD133) and progenitor cell markers (A2B5, O4). CD45 was used as a microglial marker. The A2B5+ population has consistently accounted for a large percentage of the total tumor cells (ranging from 21% to 90% of the CD45− population). When equal amounts of tissue (2 g) from glioblastoma and non-neoplastic adult human temporal lobe white matter were compared, the A2B5 fraction was greater in the glioblastomas tissue both as a percentage of all cells counted (43% vs. 8%) and in terms of absolute number of cells (4.3×10⁶ vs. 8×10³) (FIG. 33). Thus, the glioblastomas were largely composed of a massive expansion of A2B5+ cells. All glioblastomas also contained O4+ cells, but the size of the population tended to be smaller than that seen with A2B5 (ranging from 8% to 85% of the cells) (FIG. 34). Only 8 of 11 glioblastomas contained detectable numbers of CD133+ cells, ranging from 15% to 45% of the cells. When present, the CD133+ cells are a subset of a larger A2B5 population.

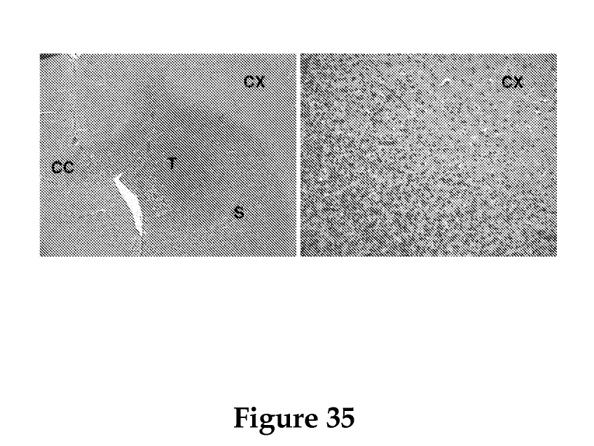

Experiments have been designed to test if the different cell populations (A2B5+/CD133+, A2B5+/CD133− and A2B5−/CD133−) contain cells with the capacity to form tumors. These cell populations have been FACS sorted from 7 human glioblastoma specimens and transplanted into the subcortical white matter of adult nude rats (2-3 rats per group and 10⁵ cells per rat). Eight animals have also been injected with unsorted cells from 3 different human glioblastomas. These studies are ongoing and many animals are still alive and have not yet been analyzed. However, animals from several groups have started to form tumors. Tumors have developed in animals injected with unsorted cells, A2B5+/CD133− cells and A2B5−/CD133− cells. The tumors from each group have shown similar histologic features; they are highly infiltrative, invading across the corpus callosum into the contralateral hemisphere (FIG. 35). Immunohistochemical stains for human nuclear antigen and K167 have shown that the human glioma cells are highly proliferative and widely dispersed. There has been significant variability between human specimens in the time it takes for the sorted cells to form tumors (2 to 5 months), but relatively little variability between animals injected with cells from a single tumor specimen.

The results presented in this Example differ from the idea that CD133 can be used to identify a subpopulation of “cancer stem cells” present in all human glioblastomas (Singh et al. 2004b). At least some human glioblastomas contain a population of A2B5+/CD133− cells that can form tumors when transplanted into nude rats. Similarly, some glioblastoma contain A2B5− cells that can also form tumors. The results presented here show that glioblastomas are composed of mixed populations of phenotypically distinct tumor cells and that cells in the different populations retain the capacity to form tumors. Glioma cells possess a high degree of plasticity with regards to the expression of phenotypic markers such as CD133, A2B5 and O4. For example, glioma cells may convert from A2B5+ to A2B5− and back, depending on the growth factor stimulation provided by the local environment, just as glial progenitors have been shown to do in culture (Gard and Pfeiffer 1993) (Avossa and Pfeiffer 1993) (Mason and Goldman 2002).

### Experimental Methods

Retrovirus production and injections: Five retroviral constructs are used: pNIT-GFP, pQ-PDGFB-HA-IRES-GFP, pQ-PDGFB-HA-IRES-DsRed, CLE-GFP and CLE-EGFR-GFP. Replication deficient virus can be generated by co-transfecting gp293 cells with the viral plasmids and VSVg. The viruses can then be injected into the adult rat white matter (stereotactic coordinates relative to bregma; 2 mm lateral, 2.5 mm rostral, 3.5 mm deep). When injected into adult white matter these viruses will predominantly infect adult glial progenitors. At 3 dpi over 80% of the infected cells are NG2+/PDGFRα+/olig2+/GFAP− (Assanah et al., 2006). These retroviruses can also be used to infect freshly isolated cells in culture. Almost 100% infectivity of isolated glial progenitors can be achieved from adult rat white matter if they are grown in growth factor containing media. If lower infection efficiency occurs with the human cells, then the human cells can be labeled using a GFP expressing lentivirus FUGW (Lois et al. 2002).

Time-lapse microscopy of slice cultures: At specified times after injection with retrovirus or cells the animals are decapitated and 300 um thick brain slices are generated as described (Farin et al. 2006). Images can be captured every 3 minutes for up to 72 hours. Tracking of cell movement and quantification of cell speed and direction can be performed using the DIAS image analysis system (Soll Technologies Inc.). With the motorized stage, multiple sections can be monitored by time-lapse, thus increasing the number of experimental conditions that can be tested. Sections can be monitored for 6 hours prior to treatment with growth factors or inhibitors to establish the baseline behavior of the cells.

Primary cultures of glial progenitors and glioma cells: White matter progenitors can be isolated from adult Sprague Dawley rats as previously described (Mason and Goldman 2002). Human cells can be isolated from surgical specimens from temporal lobe resections for epilepsy or glioma resections using the same isolation protocol. In some cases the cells may be expanded in B104 conditioned media, as described (Canoll et al. 1996) for 5 days before infection. In other cases the cells may be infected after 1 day.

Flow cytometry and FACS: Freshly isolated cells can by stained with the appropriate fluorescence-conjugated antibodies: anti-CD133, anti-A2B5, anti-04 and anti-CD45, and flowed using a FACScalibur flowcytometer or sorted using FACSaria cell sorter. Results can be analyzed using FlowJo software.

Histology and Immunohistochemistry: Brains can be fixed by perfusion with 4% paraformaldehyde and either cut on a cryostat into 10 um sections or paraffin embedded and cut on a microtome into 5 um sections. Immunofluorescence analysis can be performed on 10 μm cryosections as described (Assanah et al., 2006) using the following antibodies: anti-Olig2; anti-Nestin (1:200 mouse anti-rat 401, Chemicon, Temecula, Calif.); anti-GFAP (1:500 rabbit, DAKO, Carpinteria, Calif.); anti-GFP (1:500 rabbit, Molecular Probes, Eugene, Oreg.); anti-DsRed (1:100 rabbit, BD Biosciences, Palo Alto, Calif.); anti-HA (1:100 mouse, Covance, Berkeley, Calif.); anti-Ki67 (1:1,000 rabbit, NovoCastra, New Castle, UK); anti-PDGFRα (1:80 goat, NeoMarkers, CA), anti-NG2; SMA (DAKO, Carpinteria, Calif.); anti-CC1 (1:50 mouse, Calbiochem, Germany). The tissue can also be stained with phospho-specific antibodies against PDGFRα and EGFR and downstream targets to characterize the activation of the downstream signaling pathways. There are a number of commercially available phospho-specific antibodies that that have been shown to work well on tissue sections (Mandell 2003). A partial list of available antibodies includes Phospho-PDGFR B (Y751, Y716), EGFR (Y 1045, Y1068, Y845, Y992), Phospho-RAF (S259, S338), phospho-AKT (S473 and T308), phoshoS6 kinase (T389, T421, T412, S424), mTOR (S2448, S2481).

Small Molecule Inhibitors: There are a growing number of compounds that have been shown to inhibit specific molecules in the growth factor signaling pathways, and many of these drugs are being considered as potential therapeutic agent for the treatment of malignant gliomas (Rich and Bigner 2004). There are small molecule inhibitors commercially available for the targets considered by this Example. FIG. 23 provides a list of non-limiting examples of candidate compounds that can be used in the methods of the invention.

Human Tissue Samples and Animals: The experiments described can use fresh, frozen and paraffin embedded human tissue from surgical resection specimens obtained during surgeries, in accord with any applicable regulations. Rats can be used in the generation of the in vivo glioma models and for the slice culture experiments. The animals can be subjected to surgical procedures during the stereotactic injection of retrovirus and cells and during the placement of intracerebral catheters for intratumoral convection enhanced delivery of drugs.

### Example 4

### Characterization of Growth Factor Responsiveness and Tumorigenic Potential of Adult Glial Progenitors

Glial progenitors in the adult rat white matter can be infected with retroviruses that express growth factor (PDGF) or growth factor receptors (EGFR-GFP) and one can characterize the effects on migration and proliferation in vivo and in slice culture. The slices can also be treated with exogenous growth factors to characterize the acute effects on retrovirally labeled progenitor. The downstream signaling pathways that are activated by growth factor stimulation can be studied using phospho-specific antibodies against specific signaling molecules. Example 1 shows that adult glial progenitors have a remarkable capacity to migrate, proliferate and form tumors when stimulated by constitutive growth factor signaling. This Example further defines the type of cells that are giving rise to tumors and characterize their response to growth factors in vivo and in slice culture.

### Characterization of the Tumorigenic Potential of A2B5+ and O4+ Cells from Adult Rat and Human White Matter Using the PDGF Expressing Retrovirus

Glial progenitors in the adult rat white matter are a heterogeneous population of cells. This heterogeneity can be accounted for, at least in part, by cells being in different stages of differentiation along the oligodendrocyte lineage. With regards to the progenitor cell markers A2B5+ and O4+, there are 3 distinct populations: A2B5+/O4−, A2B5+/O4+ and A2B5−/O4+(Mason and Goldman 2002) (Gensert and Goldman 2001). Studies were designed to determine whether the capacity for glial progenitors to revert from a less migratory/more mature stage (O4+) to a more migratory/less mature stage (A2B5+) also pertains to their tumorigenic potential. A2B5+/O4− and A2B5−/O4+ cells can be isolated from adult rat white matter and infected in vitro with the PDGF expressing retrovirus provided by the invention and then transplanted into adult brain (as described for unsorted cells in Assanah et al., 2006). Flow cytometry can be performed on tumors that form from the sorted cells to assess the co-expression of GFP, O4 and A2B5.

Similar experiments can be performed on glial progenitors (A2B5+, O4+ and unsorted) isolated from human temporal lobe white matter from surgical resections for temporal lobe epilepsy. These experiments can be designed to test whether human glial progenitors resemble rat progenitors with regards to their tumorigenic potential when infected with the PDGF-IRES-DsRed expressing retrovirus. Results have shown that these retroviruses can infect unsorted human glial progenitor cells with high efficiency. Human progenitors can also be infected with control retrovirus (pNIT-GFP) to characterize their normal behavior when transplanted into nude rats. Some animals can be used to make slice cultures to characterize the effects of growth factor stimulations on migration and proliferation of human glial progenitor by time-lapse microscopy.

### Characterization of the Effects of EGFR-GFP Expression on Adult Glial Progenitors

Constitutive expression of EGFR-GFP keeps neonatal glial progenitor cells immature, migratory and proliferative for up to 15 wpi. The immunophenotype of EGFR-GFP infected cells is identical to that of the PDGF infected cells with respect to tested markers (NG2+/PDGFRα+/olig2+/GFAP−/CC1−), showing that this immunoprofile is characteristic of a common state of growth factor activated glial progenitors. EGFR is down-regulated during normal brain development (Fox and Kornblum 2005, Seroogy et al. 1995), whereas PDGFRα is widely expressed by adult glial progenitors (Dawson et al. 2003, Horner et al. 2002, Nishiyama et al. 1996). These findings show that there may be a shift from EGF-responsive progenitors to predominantly PDGF-responsive progenitors during postnatal gliogenesis (Gago et al. 2003). However, a residual population of EGF responsive cells may exist in the adult SVZ; injecting EGF ligand into the lateral ventricle induces a marked proliferation of cells that migrate out of the SVZ into the striatum and white matter (Craig et al. 1996) (Doetsch et al. 2002). The resulting lesions superficially resemble the beginnings of a glioma.

Adult rat white matter (forceps minor of the corpus callosum) can be injected with EGFR-GFP expressing retrovirus or control virus CLE-GFP (10⁵ CFUs in 1 μl DMEM). One can target the same population of adult white matter progenitors that give rise to gliomas when infected with the PDGF virus (NG2+/olig2+/PDGFRα+/GFAP−). The animals can be sacrificed at 3, 10, and 30 dpi and 6 months post injection and perform immunofluorescence analysis on fixed cryosections to determine the distribution, proliferation rate (KI67) and marker profile (NG2/PDGFRα/olig2/GFAP) of the GFP+ cells. These sections can also be used for immunohistochemical analysis using phospho-specific antibodies against EGFR and downstream signaling molecules to characterize which signaling pathways are being activated. In the neonate experiments, the migration of EGFR-GFP cells was predominantly via white matter tract. If adult glial progenitors behave similarly, this should lead to diffuse hyper-cellularity of the white matter, and eventually tumor formation. However, results show that tumor formation, when it does occur, takes several months. This very slow onset relative to the PDGF model reflects that the EGFR-GFP is stimulating cell proliferation and migration in a primarily cell-autonomous manner whereas PDGF drives tumor growth via both autocrine and paracrine stimulation. However, EGFR and PDGFR signaling may have distinctly different effects on glial progenitor migration and proliferation. For example, EGFR may predominantly drive cell migration whereas PDGF signaling may have a greater effect on proliferation.

Slice cultures can be generated at 10 dpi and time-lapse microscopy can be performed to monitor migration and proliferation of EGFR-GFP+ cells. One advantage of using the EGFR-GFP fusion protein is that time-lapse microscopy can be used to visualize the distribution of the receptor as the cells migrate through the brain tissue. Time-lapse studies show that the receptor accumulates in the leading process, suggesting a role in directed migration. The distribution of EGFR may be affected by the distribution and abundance of ligands in the environment. In vitro experiments have shown that ligand can induce receptor clustering—a first step in signaling (Ichinose et al. 2004). Using time-lapse, experiments can be designed to test if adding exogenous EGF ligand (10 ng/ml) to the slice culture medium will induce receptor clustering and characterize how these events correlate with cell process extension/retraction, migration and division. At the end of the time-lapse experiments the slices can be fixed and stained with K167 to determine proliferation index and with anti-phospho-EGFR and other phospho-specific antibodies to characterize which downstream pathways are activated by EGF stimulation.

### Characterization of the Short-Term Effects of PDGF and EGF on Adult Glial Progenitor Migration And Proliferation by Treating Slice Cultures with Soluble Ligands

Adult glial progenitors are normally non-migratory and slowly proliferating. The retrovirus studies presented in the Examples have shown that constitutive growth factor stimulation will drive these cells to migrate and proliferate. However, it takes up to 3 days before the retrovirus-infected cells can be detected and by this time the infected cells may already be activated and many interesting changes may have already occurred. The methods of the invention can be used to study the early effects of growth factor stimulation and how this leads to the transformation of slowly proliferating/non-migratory cells to rapidly proliferating/migratory cells that closely resemble glioma cells. Glial progenitors in the adult subcortical white matter can be infected with control GFP expressing retrovirus; slice cultures can be generated at 3 dpi; the slices can be treated with soluble growth factors (PDGF and EGF alone and in combination); and the effects on the migration and proliferation of GFP+ cells can be monitored by time-lapse microscopy. The slices may be monitored by time-lapse microscopy for up to about 72 hours. Cell migration can be tracked using metamorph and DIAS image analysis system as described (Farin et al. 2006). At the end of the time-lapse experiments the slices can be fixed in 4% paraformaldehyde and stained for phosphohistone H3 and/or K167 to determine the proliferation index or with phospho-specific antibodies to characterize the activation state of receptors and down-stream signaling molecules.

Results indicate that untreated cells will be non-migratory and non-proliferative and that PDGF treatment will stimulate them to begin migrating and dividing. This response may take several hours, or even days, if it requires a complex reprogramming of the cell's state, involving the expression of new proteins. Alternatively, the adult progenitors may be competent to respond, in which case they may begin migrating and proliferating almost immediately. The adult progenitors are expected respond to PDGF since they are known to express PDGFRα. The infected cells may respond differently to PDGF and EGF. For example, PDGF may elicit a more proliferative response and EGF a more migratory response. If so, the methods of the invention can be used to look for the differences in the down-stream signal events that account for the different cellular responses. Similar sets of experiments can be performed on slice cultures generated at 10 dpi and 30 dpi. Studies show that by 10 dpi most of the adult glial progenitors infected with control virus had acquired a complex multipolar morphology and expressed CC1 (a marker of more mature oligodendroglia). Studies can be designed to determine whether the more differentiated appearing cells will still respond to growth factors. It may be that the more differentiated cells will need to revert to a less differentiated state, and therefore take longer to begin migrating and proliferating in response to growth factor. Or, they may not respond at all. The methods provided by the invention can also be used test the effects of different concentrations of growth factors. In vitro studies on oligodendroglial progenitors show that low and high PDGF concentrations activate distinct downstream signals and induced different cellular responses, showing that PDGF signaling acts more like a graded rheostat than an “ON-OFF” switch (McKinnon et al. 2005). Studies can be designed to test if a similar type of graded response occurs in adult progenitor in the context of living brain tissue.

### Characterization the Activation of Receptors and Down-Stream Signaling Molecules

PDGFR and EGFR activate a number of second messenger pathways (FIG. 23). To characterize which pathways are activated by the growth factor stimulated cells, immunohistochemistry can be performed using phospho-specific antibodies against several molecules, for example, in the PI3K-AKT and RAS-MAPK pathways. The immunohistochemical analysis can be used to provide cellular-spatial information about which signaling pathways are activated in specific subpopulations of cells or certain areas of the tumor. For example, anti-phospho-S6K strongly stains the majority of cells in the PDGF driven tumor and in a subpopulation of cells infiltrating the white matter, but does not stain areas of pseudopalisading necrosis. For more quantitative analysis, western blot analysis can be performed using the same antibodies when possible. To characterize the activation state of these signaling molecules, one can immunoprecipitate with anti-phosphotyrosine and blot with the receptor-specific antibodies. For example, this analysis can be performed on cryosections of tissue from the PDGF driven tumors. Similar analyses can be performed on cryosections of brains infected with the EGFR-GFP expressing retroviruses, where one can look for co-localization of EGFR-GFP fusion protein with immunoreactivity of the various phospho-specific antibodies. Co-localization of GFP and phospho-EGFR would show that EGFR-GFP was being activated.

### Example 5

### Characterization of Growth Factor Responsiveness and Tumorigenic Potential of Progenitor-Like Cells Isolated from Human Gliomas

Human glioblastomas contain an abundance of cells that resemble glial progenitors, including A2B5+ and O4+ cells. One can isolate subpopulations of glioma cells with progenitor-like phenotype using FACS for glial progenitor markers (A2B5 and O4), infect the cells with GFP expressing retrovirus, and transplant them into nude rats to determine their tumorigenic potential. Time-lapse microscopy can be used to characterize the migratory and proliferative behavior of the GFP−tagged glioma cells and their response to growth factors in slice culture generated from the xenografts.

### Characterization of the Tumorigenic Potential of Different Subpopulations of Progenitor-Like Cells in Human Gliomas

Using FACS, cells have been isolated on the basis of markers for stem cells (CD133) and progenitor cells (A2B5 and O4) and transplanted into nude rats. Results show that A2B5+ cells have the capacity to give rise to tumors with a frequency similar to that seen with unsorted cells. The tumors that form show many of the histologic features of the initial tumors. They also closely resemble the tumors formed by the PDGF expressing retrovirus. They are highly infiltrative, invading the cortex and crossing the corpus callosum into the contralateral hemisphere. Double immunofluorescence analysis for human nuclear antigen and K167 shows that the human glioma cells are highly proliferative and widely dispersed among the host rat cells. These experiments are ongoing and we will expand our analysis to test if O4+ cells isolated from human gliomas are also tumorigenic when injected into nude rats. The different populations (A2B5+ and O4+) may each contain a mixture of recruited progenitors and true glioma cells and that both glioma cells and progenitor cells can to revert back and forth between these different stages of differentiation depending on the growth factor simulation of the local environment. To address this, the freshly sorted human glioma cells can be labeled with GFP expressing retrovirus and then transplanted into nude rats. If tumors form, flow cytometry can be performed to look for co-expression of GFP, A2B5 and O4. A finding that A2B5+ glioma cells give rise to tumors that contain O4+/GFP+ cells, would show that some degree of differentiation can occur within the tumors. Conversely, a finding that O4+ glioma cells give rise to tumors that contain significant numbers of A2B5+/GFP+ cells, would show that glioma cells can revert back and forth between states.

### Characterize the Migratory and Proliferative Behavior and Growth Factor Responsiveness of Human Glioma Cells in Slice Cultures

Time-lapse microscopy can be used to monitor the migration and proliferation of GFP tagged human glioma (sorted and unsorted) in slice cultures generated from the xenografts. Time-lapse microscopy can be performed on untreated cells to establish a level of basal behavior. The human glioma cells are expected to migrate and proliferate in a manner that is similar to that seen with the PDGF glioma model and with transplanted C6 glioma cells (Farin et al. 2006). The cells are also expected to have a bipolar morphology and migrate in a saltatory manner. Based on the results looking at the distribution of transplanted glioma cells by immunohistochemistry for human nuclear antigen, one expects that the GFP+ glioma cells will predominantly migrate along white matter tracts and along the abluminal surface of blood vessels. DIAS can be used to track cell paths and measure the speed and directionality of migration. This quantitative analysis can serve as a basis with which to compare the effects of the growth factors and inhibitors.

The effects of exogenous PDGF and EGF (alone or in combination) can also be tested on the cells in slice. Dose response studies can be performed to look for a graded response. Some glioma cells are expected to be stimulated to migrate and proliferate more rapidly when they are treated with exogenous growth factor. The response will depend on whether the cells are expressing the appropriate growth factor and growth factor receptors. Most human glioblastomas express PDGFR, EGFR or both. Some gliomas also secrete the growth factors allowing for autocrine and paracrine signaling and/or express constitutively active mutants (such as EGFRvIII), and one would expect these cells to have elevated levels of growth factor signaling even in the absence of exogenous ligands. Cell culture experiments have shown that most glioma cell lines respond to exogenous ligand (Pollack et al. 1991) (Pedersen et al. 1994) and one expects that most xenografts will show some response. At the end of the time-lapse analysis the slices can be fixed in 4% paraformaldehyde and immunofluorescence analysis can be performed to determine the proliferation index (KI67). The expression of growth factors and receptors can be characterized and the phosphorylation status of growth factor receptors and downstream signaling pathways using phospho-specific antibodies can also be characterized.

### Characterization of the Relationship Between Growth Factor Signaling and Glioma Grade

In addition to glioblastomas, the methods of the invention can be used to analyze lower grade astrocytomas and oligodendrogliomas. Eight low-grade gliomas (4 astrocytomas and 4 oligodendrogliomas) have been analyzed. Low-grade gliomas contain a higher percentage of O4+ cells. This shows that low-grade gliomas may be composed of cells with a more differentiated phenotype and this may correlate with a less aggressive growth behavior. The phenotype and the behavior of the cells may be strongly influenced by the level of growth factor stimulation from the environment.

To test the tumorigenic potential of the different subpopulations of cells in low-grade gliomas, cells (sorted and unsorted) can be infected with GFP expressing virus and around 10⁵ cells from each group can be transplanted into nude rats. For the first 5 tumors analyzed, all animals can be followed until the show signs of tumor morbidity. It is possible that tumors will take longer to form with these cells than they do with cells from glioblastomas. If no animals form tumors by 6 months post injection then the animals can be sacrificed and histological and immunofluorescence analyses can be performed to look for tumor formation and to characterize the number, distribution and proliferation rate of GFP+ cells.

The behavior of cells in slice cultures generated from these brains can be monitored at 10 dpi and at the time of tumor formation. One can predict the following: 1) low-grade glioma cells will behave more like normal glial progenitors than do high grade glioma cells, and 2) low grade glioma cells can be driven to behave more aggressively in response to growth factor stimulation. Such findings would show that higher levels of growth factor signaling can drive the formation of more malignant tumors (Shih et al. 2004). Histological and immunohistochemical analyses can be performed on cryosections and/or paraffin embedded section from surgical specimen from which the primary human glioma cells are isolated. This will allow one to correlate results with specific grades and subtypes of gliomas and with the expression of growth factors and growth factor receptors that are found in the original tumor.

The onset of tumor formation can vary from 2 months to more than 5 months post-transplantation. However, the formation of tumors between animals injected with cells from the same human tumor has been more consistent, with all animals developing tumor-induced morbidity within a week of each other. Enough cells can be isolated to inject around 3 animals in each group.

### Example 6

### Effects of Blocking Growth Factor Signaling on Migration, Proliferation and Survival of Glial Progenitors and Glioma Cells

A pharmacologic approach can be used to inhibit growth factor signaling in the retrovirus induced tumors. Slice cultures generated from the PDGF driven tumors can be treated with small molecule inhibitors (alone and in combinations). As discussed above, the slice culture system has the advantage that it allows easy delivery of small molecules at defined concentrations. The slices can also be washed to test if the effects are reversible. Furthermore, time-lapse microscopy can be used to directly monitor the effects of these molecules on the migration and proliferation of the GFP+ and/or DsRed+ cells within the microenvironment of living brain tissue. Drugs and combinations of drugs that show promising effects in the slice cultures can be tested in vivo using intracerebral convection enhanced delivery (CED).

### Characterization of the Effects of Small Molecule Inhibitors on Migration, Proliferation and Survival in Slice Culture

The effects of the PDGFR inhibitor GLEEVEC (Novartis) on the PDGF driven tumors can be assessed using the methods of the invention. This glioma model tumor formation is driven primarily by PDGF signaling. Therefore, blocking PDGFR activity should have an inhibitory effect on the growth and migration of the PDGF driven tumors. These tumors may upregulate the expression other growth factor and/or growth factor receptors. Although, high levels of EGFR expression in the PDGF driven tumor are not seen by immunohistochemical analysis. Other studies found that treating PDGF driven tumors with the PDGFR inhibitor PTK787 caused high-grade tumors to acquire low-grade histology, but it did not completely eradicate tumor growth (Shih et al. 2004). Such results show the importance of testing combinations of inhibitors and of targeting down-stream signaling molecules that may act as nodal point in growth factor signaling cascades.

The effects of inhibitors of down-streams signaling molecules can also be tested, for example, the PI3K-AKT and MAP kinase pathways (FIG. 23). Each of these pathways are activated by both PDGFR and EGFR and they are well established to mediate effects on cell migration, proliferation and survival in a number of cell types including gliomas (Chandrasekar et al. 2003, Gu et al. 1998, Koul et al. 2006, Lefranc et al. 2005). The details of the cellular responses associated with each of these pathways are context specific and likely depends on the cell type and environmental factors (Sinor and Lillien 2004, Steinbach et al. 2004). A recent study showed that combined targeting of PI3K/AKT and RAS/MAP pathways has a synergistic inhibitory effect on the growth of human glioma cell lines in vitro (Edwards et al. 2006). The results from such in vitro studies can be used to determine which combinations and concentration of drugs to test in the system provided by the invention.

Slices generated from tumors at 10 dpi can be monitored by time-lapse for 6 hours prior to drug treatment to establish baseline behavior, and then drugs will be added to the slice culture medium and the slices will be monitored for around 6-72 hours. The time-lapse analysis can focus on cell migration and proliferation, as above. Studies have shown that the death of individual cells can be seen during time-lapse as a loss of GFP fluorescence. This is a rare event under control conditions, but may be much more common when the slices are treated with inhibitors. As a measure of slice viability, one can count the number and percentage of GFP+ cells that “blink off” during the course of a time-lapse. At the end of the treatment the slices can be fixed and stained for K167 to look for effects on cell proliferation and phospho-specific antibodies can be used to look for direct evidence of inhibition of specific signaling molecules. TUNEL can be performed to look for effects on cell survival.

These analyses can also be used to determine which signaling pathways mediate specific cellular responses. Previous studies have shown that PDGF and EGF are pleotrophic, affecting migration, proliferation and survival to varying degrees depending on the cell type studied. It is not known at what point the signaling pathways that drive migration and proliferation diverge. It is likely that some inhibitors will preferentially affect migration and others proliferation. Time-lapse analysis will allow us to directly monitor these behaviors at the cellular level and thus detect an effect on one or the other behavior. Effects on cell viability will also affect migration and proliferation. In some experiments, the inhibitors can be removed and the slices can be rinsed to look for reversibility of effects. If cells recover after prolonged exposure it would show that the effect was cytostatic rather than cytotoxic. Methods of the invention can be used to determine how various drugs affect specific cellular responses and to help design more effective drug combinations.

### Treating the Retroviral Tumors by CED of Small Molecule Inhibitors

Techniques have been developed to deliver small molecules directly to the tumor via intracerebral catheters. An advantage of this approach is that it avoids problems such as systemic toxicity and inhibition of drugs reaching the tumors by the blood brain barrier (Chamberlain 2006). Because the PDGF retrovirus drives tumor formation in a rapid and consistent manner (similar to that seen with the transplantation of glioma cell lines), one of ordinary skill in the art would know how to transfer the technology to this model. The catheter can be implanted at the injection site at 7 dpi. Results show that 100% of the animals have small tumors at this time point. A small pump can be implanted under the skin and the drugs are perfused at about 1 μl per minute over the course of 7 days. On the last day of treatment (14 dpi), 6 rats from each group can be sacrificed and analyzed histologically and immunohistochemically to characterize the effects on cell proliferation, dispersion and survival. GFP will allow one to identify the number and distribution of retrovirus-infected cells. K167 can be used to assess proliferation and TUNEL analysis can be performed to determine the level of programmed cell death of GFP+ cells. Immunohistochemical analysis with phospho-specific antibodies can be performed to characterize the effects on receptor phosphorylation and downstream signaling. One can follow the remaining animals in each group until they show signs of tumor-induced morbidity, at which time the animals can be sacrificed and analyzed as above. These survival studies can also be used to provide information on the toxicity of drugs and combinations of drugs. Treatment regimes that show promise can be brought to clinical trial.

### REFERENCES

Aguirre, A., T. A. Rizvi, N. Ratner, and V. Gallo. 2005. Overexpression of the epidermal growth factor receptor confers migratory properties to nonmigratory postnatal neural progenitors. J Neurosci 25: 11092-106.
Avossa, D., and S. E. Pfeiffer. 1993. Transient reversion of O4+GalC-oligodendrocyte progenitor development in response to the phorbol ester TPA. J Neurosci Res 34: 113-28.
Assanah M, Lockhead, R. Ogden, A., Bruce, J., Goldman, J., Canoll, P., 2006. Glial progenitors in adult white matter are driven to form malignant gliomas by Platelet-Derived Growth Factor-expressing Retroviruses. J. Neurosci 26(25). June 21.
Bouvier, C., C. Bartoli, L. Aguirre-Cruz, I. Virard, C. Colin, C. Fernandez, J. Gouvernet, and D. Figarella-Branger. 2003. Shared oligodendrocyte lineage gene expression in gliomas and oligodendrocyte progenitor cells. J Neurosurg 99: 344-50.
Brockmann, M. A., U. Ulbricht, K. Gruner, R. Fillbrandt, M. Westphal, and K. Lamszus. 2003. Glioblastoma and cerebral microvascular endothelial cell migration in response to tumor-associated growth factors. Neurosurgery 52: 1391-9; discussion 1399.
Bruce, J. N., A. Falavigna, J. P. Johnson, J. S. Hall, B. D. Birch, J. T. Yoon, E. X. Wu, R. L. Fine, and A. T. Parsa. 2000. Intracerebral clysis in a rat glioma model. Neurosurgery 46: 683-91.
Canoll, P. D., J. M. Musacchio, R. Hardy, R. Reynolds, M. A. Marchionni, and J. L. Salzer. 1996. GGF/neuregulin is a neuronal signal that promotes the proliferation and survival and inhibits the differentiation of oligodendrocyte progenitors. Neuron 17: 229-43.
Chamberlain, M. C. 2006. Treatment options for glioblastoma. Neurosurg Focus 20: E2.
Chandrasekar, N., S. Mohanam, M. Gujrati, W. C. Olivero, D. H. Dinh, and J. S. Rao. 2003. Downregulation of uPA inhibits migration and PI3k/Akt signaling in glioblastoma cells. Oncogene 22: 392-400.
Chekenya, M., and G. J. Pilkington. 2002. NG2 precursor cells in neoplasia: functional, histogenesis and therapeutic implications for malignant brain tumours. J Neurocytol 31: 507-21.
Craig, C. G., V. Tropepe, C. M. Morshead, B. A. Reynolds, S. Weiss, and D. van der Kooy. 1996. In vivo growth factor expansion of endogenous subependymal neural precursor cell populations in the adult mouse brain. J Neurosci 16: 2649-58.
Dai, C., J. C. Celestino, Y. Okada, D. N. Louis, G. N. Fuller, and E. C. Holland. 2001. PDGF autocrine stimulation dedifferentiates cultured astrocytes and induces oligodendrogliomas and oligoastrocytomas from neural progenitors and astrocytes in vivo. Genes Dev 15: 1913-25.
Dawson, M. R., J. M. Levine, and R. Reynolds. 2000. NG2-expressing cells in the central nervous system: are they oligodendroglial progenitors? J Neurosci Res 61: 471-9.
Dawson, M. R., A. Polito, J. M. Levine, and R. Reynolds. 2003. NG2-expressing glial progenitor cells: an abundant and widespread population of cycling cells in the adult rat CNS. Mol Cell Neurosci 24: 476-88.
Doetsch, F., L. Petreanu, I. Caille, J. M. Garcia-Verdugo, and A. Alvarez-Buylla. 2002. EGF converts transit-amplifying neurogenic precursors in the adult brain into multipotent stem cells. Neuron 36: 1021-34.
Edwards, L. A., M. Verreault, B. Thiessen, W. H. Dragowska, Y. Hu, J. H. Yeung, S. Dedhar, and M. B. Bally. 2006. Combined inhibition of the phosphatidylinositol 3-kinase/Akt and Ras/mitogen-activated protein kinase pathways results in synergistic effects in glioblastoma cells. Mol Cancer Ther 5: 645-54.
Farin, A., S. O. Suzuki, M. Weiker, J. E. Goldman, J. N. Bruce, and P. Canoll. 2006. Transplanted glioma cells migrate and proliferate on host brain vasculature: a dynamic analysis. Glia 53: 799-808.
Fox, I. J., and H. I. Kornblum. 2005. Developmental profile of ErbB receptors in murine central nervous system: implications for functional interactions. J Neurosci Res 79: 584-97.
Frappaz, D., S. E. Singletary, G. Spitzer, and A. Yung. 1988. Enhancement of growth of primary metastatic fresh human tumors of the nervous system by epidermal growth factor in serum-free short term culture. Neurosurgery 23: 355-9.
Gago, N., V. Avellana-Adalid, A. B. Evercooren, and M. Schumacher. 2003. Control of cell survival and proliferation of postnatal PSA-NCAM(+) progenitors. Mol Cell Neurosci 22: 162-78.
Gard, A. L., and S. E. Pfeiffer. 1993. Glial cell mitogens bFGF and PDGF differentially regulate development of O4+GalC-oligodendrocyte progenitors. Dev Biol 159: 618-30.
Gensert, J. M., and J. E. Goldman. 2001. Heterogeneity of cycling glial progenitors in the adult mammalian cortex and white matter. J Neurobiol 48: 75-86.
Gil-Perotin, S., M. Marin-Husstege, J. Li, M. Soriano-Navarro, F. Zindy, M. F. Roussel, J. M. Garcia-Verdugo, and P. Casaccia-Bonnefil. 2006. Loss of p53 induces changes in the behavior of subventricular zone cells: implication for the genesis of glial tumors. J Neurosci 26: 1107-16.
Graves, L. M., J. Han, and H. S. Earp, 3rd. 2002. Transactivation of the EGF Receptor: Is the PDGF Receptor an Unexpected Accomplice? Mol Interv 2: 208-12.
Gu, J., M. Tamura, and K. M. Yamada. 1998. Tumor suppressor PTEN inhibits integrin- and growth factor-mediated mitogen-activated protein (MAP) kinase signaling pathways. J Cell Biol 143: 1375-83.
Hermanson, M., K. Funa, M. Hartman, L. Claesson-Welsh, C. H. Heldin, B. Westermark, and M. Nister. 1992. Platelet-derived growth factor and its receptors in human glioma tissue: expression of messenger RNA and protein suggests the presence of autocrine and paracrine loops. Cancer Res 52: 3213-9.
Hesselager, G., and E. C. Holland. 2003. Using mice to decipher the molecular genetics of brain tumors. Neurosurgery 53: 685-94; discussion 695.
Hesselager, G., L. Uhrbom, B. Westermark, and M. Nister. 2003. Complementary effects of platelet-derived growth factor autocrine stimulation and p53 or Ink4a-Arf deletion in a mouse glioma model. Cancer Res 63: 4305-9.
Holland, E. C., W. P. Hively, R. A. DePinho, and H. E. Varmus. 1998. A constitutively active epidermal growth factor receptor cooperates with disruption of G1 cell-cycle arrest pathways to induce glioma-like lesions in mice. Genes Dev 12: 3675-85.
Hommes, O. R., and C. P. Leblond. 1967. Mitotic division of neuroglia in the normal adult rat. J Comp Neurol 129: 269-78.
Horner, P. J., M. Thallmair, and F. H. Gage. 2002. Defining the NG2-expressing cell of the adult CNS. J Neurocytol 31: 469-80.
Ichinose, J., M. Murata, T. Yanagida, and Y. Sako. 2004. EGF signalling amplification induced by dynamic clustering of EGFR. Biochem Biophys Res Commun 324: 1143-9.
Ignatova, T. N., V. G. Kukekov, E. D. Laywell, O. N. Suslov, F. D. Vrionis, and D. A. Steindler. 2002. Human cortical glial tumors contain neural stem-like cells expressing astroglial and neuronal markers in vitro. Glia 39: 193-206.
Joensuu, H., M. Puputti, H. Sihto, O. Tynninen, and N. N. Nupponen. 2005. Amplification of genes encoding KIT, PDGFRalpha and VEGFR2 receptor tyrosine kinases is frequent in glioblastoma multiforme. J Pathol 207: 224-31.
Kaiser, M. G., A. T. Parsa, R. L. Fine, J. S. Hall, I. Chakrabarti, and J. N. Bruce. 2000. Tissue distribution and antitumor activity of topotecan delivered by intracerebral clysis in a rat glioma model. Neurosurgery 47: 1391-8; discussion 1398-9.
Kakita, A., and J. E. Goldman. 1999. Patterns and dynamics of SVZ cell migration in the postnatal forebrain: monitoring living progenitors in slice preparations. Neuron 23: 461-72.
Koul, D., R. Shen, S. Bergh, X. Sheng, S. Shishodia, T. A. Lafortune, Y. Lu, J. F. de Groot, G. B. Mills, and W. K. Yung. 2006. Inhibition of Akt survival pathway by a small-molecule inhibitor in human glioblastoma. Mol Cancer Ther 5: 637-44.
Lammering, G., P. S. Lin, J. N. Contessa, J. L. Hampton, K. Valerie, and R. K. Schmidt-Ullrich. 2001. Adenovirus-mediated overexpression of dominant negative epidermal growth factor receptor-CD533 as a gene therapeutic approach radiosensitizes human carcinoma and malignant glioma cells. Int J Radiat Oncol Biol Phys 51: 775-84.
Lefranc, F., J. Brotchi, and R. Kiss. 2005. Possible future issues in the treatment of glioblastomas: special emphasis on cell migration and the resistance of migrating glioblastoma cells to apoptosis. J Clin Oncol 23: 2411-22.
Ligon, K. L., J. A. Alberta, A. T. Kho, J. Weiss, M. R. Kwaan, C. L. Nutt, D. N. Louis, C. D. Stiles, and D. H. Rowitch. 2004. The oligodendroglial lineage marker OLIG2 is universally expressed in diffuse gliomas. J Neuropathol Exp Neurol 63: 499-509.
Lois, C., E. J. Hong, S. Pease, E. J. Brown, and D. Baltimore. 2002. Germline transmission and tissue-specific expression of transgenes delivered by lentiviral vectors. Science 295: 868-72.
Mandell, J. W. 2003. Phosphorylation state-specific antibodies: applications in investigative and diagnostic pathology. Am J Pathol 163: 1687-98.
Mason, J. L., and J. E. Goldman. 2002. A2B5+ and O4+ Cycling progenitors in the adult forebrain white matter respond differentially to PDGF-AA, FGF-2, and IGF-1. Mol Cell Neurosci 20: 30-42.
McKinnon, R. D., S. Waldron, and M. E. Kiel. 2005. PDGF alpha-receptor signal strength controls an RTK rheostat that integrates phosphoinositol 3′-kinase and phospholipase Cgamma pathways during oligodendrocyte maturation. J Neurosci 25: 3499-508.
Nishiyama, A., X. H. Lin, N. Giese, C. H. Heldin, and W. B. Stallcup. 1996. Co-localization of NG2 proteoglycan and PDGF alpha-receptor on O2A progenitor cells in the developing rat brain. J Neurosci Res 43: 299-314.
Nunes, M. C., N. S. Roy, H. M. Keyoung, R. R. Goodman, G. McKhann, 2nd, L. Jiang, J. Kang, M. Nedergaard, and S. A. Goldman. 2003. Identification and isolation of multipotential neural progenitor cells from the subcortical white matter of the adult human brain. Nat Med 9: 439-47.
Pedersen, P. H., G. O. Ness, O. Engebraaten, R. Bjerkvig, J. R. Lillehaug, and O. D. Laerum. 1994. Heterogeneous response to the growth factors [EGF, PDGF (bb), TGF-alpha, bFGF, IL-2] on glioma spheroid growth, migration and invasion. Int J Cancer 56: 255-61.
Pollack, I. F., M. S. Randall, M. P. Kristofik, R. H. Kelly, R. G. Selker, and F. T. Vertosick, Jr. 1991. Response of low-passage human malignant gliomas in vitro to stimulation and selective inhibition of growth factor-mediated pathways. J Neurosurg 75: 284-93.
Reya, T., S. J. Morrison, M. F. Clarke, and I. L. Weissman. 2001. Stem cells, cancer, and cancer stem cells. Nature 414: 105-11.
Rich, J. N., and D. D. Bigner. 2004. Development of novel targeted therapies in the treatment of malignant glioma. Nat Rev Drug Discov 3: 430-46.
Roy, N. S., S. Wang, C. Harrison-Restelli, A. Benraiss, R. A. Fraser, M. Gravel, P. E. Braun, and S. A. Goldman. 1999. Identification, isolation, and promoter-defined separation of mitotic oligodendrocyte progenitor cells from the adult human subcortical white matter. J Neurosci 19: 9986-95.
Saito, Y., J. Haendeler, Y. Hojo, K. Yamamoto, and B. C. Berk. 2001. Receptor heterodimerization: essential mechanism for platelet-derived growth factor-induced epidermal growth factor receptor transactivation. Mol Cell Biol 21: 6387-94.
Sanai, N., A. Alvarez-Buylla, and M. S. Berger. 2005. Neural stem cells and the origin of gliomas. N Engl J Med 353: 811-22.
Savarese, T. M., T. Jang, H. P. Low, R. Salmonsen, N. S. Litofsky, Z. Matuasevic, A. H. Ross, and L. D. Recht. 2005. Isolation of immortalized, INK4a/ARF-deficient cells from the subventricular zone after in utero N-ethyl-N-nitrosourea exposure. J Neurosurg 102: 98-108.
Seroogy, K. B., C. M. Gall, D. C. Lee, and H. I. Komblum. 1995. Proliferative zones of postnatal rat brain express epidermal growth factor receptor mRNA. Brain Res 670: 157-64.
Shi, J., A. Marinovich, and B. A. Barres. 1998. Purification and characterization of adult oligodendrocyte precursor cells from the rat optic nerve. J Neurosci 18: 4627-36.
Shih, A. H., C. Dai, X. Hu, M. K. Rosenblum, J. A. Koutcher, and E. C. Holland. 2004. Dose-dependent effects of platelet-derived growth factor-B on glial tumorigenesis. Cancer Res 64: 4783-9.
Shih, A. H., and E. C. Holland. 2005. Platelet-derived growth factor (PDGF) and glial tumorigenesis. Cancer Lett.
Shoshan, Y., A. Nishiyama, A. Chang, S. Mork, G. H. Barnett, J. K. Cowell, B. D. Trapp, and S. M. Staugaitis. 1999. Expression of oligodendrocyte progenitor cell antigens by gliomas: implications for the histogenesis of brain tumors. Proc Natl Acad Sci USA 96: 10361-6.
Singh, S. K., I. D. Clarke, T. Hide, and P. B. Dirks. 2004a. Cancer stem cells in nervous system tumors. Oncogene 23: 7267-73.
Singh, S. K., I. D. Clarke, M. Terasaki, V. E. Bonn, C. Hawkins, J. Squire, and P. B. Dirks. 2003. Identification of a cancer stem cell in human brain tumors. Cancer Res 63: 5821-8.
Singh, S. K., C. Hawkins, I. D. Clarke, J. A. Squire, J. Bayani, T. Hide, R. M. Henkelman, M. D. Cusimano, and P. B. Dirks. 2004b. Identification of human brain tumour initiating cells. Nature 432: 396-401.
Sinor, A. D., and L. Lillien. 2004. Akt-1 expression level regulates CNS precursors. J Neurosci 24: 8531-41.
Steinbach, J. P., A. Klumpp, H. Wolburg, and M. Weller. 2004. Inhibition of epidermal growth factor receptor signaling protects human malignant glioma cells from hypoxia-induced cell death. Cancer Res 64: 1575-8.
Suzuki, S. O., and J. E. Goldman. 2003. Multiple cell populations in the early postnatal subventricular zone take distinct migratory pathways: a dynamic study of glial and neuronal progenitor migration. J Neurosci 23: 4240-50.
Uhrbom, L., M. Kastemar, F. K. Johansson, B. Westermark, and E. C. Holland. 2005. Cell type-specific tumor suppression by Ink4a and Arf in Kras-induced mouse gliomagenesis. Cancer Res 65: 2065-9.
Weiss, W. A., M. Israel, C. Cobbs, E. Holland, C. D. James, D. N. Louis, C. Marks, A. I. McClatchey, T. Roberts, T. Van Dyke, C. Wetmore, I. M. Chiu, M. Giovannini, A. Guha, R. J. Higgins, S. Marino, I. Radovanovic, K. Reilly, and K. Aldape. 2002. Neuropathology of genetically engineered mice: consensus report and recommendations from an international forum. Oncogene 21: 7453-63.
Westermark, B., C. H. Heldin, and M. Nister. 1995. Platelet-derived growth factor in human glioma. Glia 15: 257-63.
Wolswijk, G., and M. Noble. 1992. Cooperation between PDGF and FGF converts slowly dividing O-2Aadult progenitor cells to rapidly dividing cells with characteristics of O-2Aperinatal progenitor cells. J Cell Biol 118: 889-900.
Wolswijk, G., P. N. Riddle, and M. Noble. 1991. Platelet-derived growth factor is mitogenic for O-2Aadult progenitor cells. Glia 4: 495-503.
Wren, D., G. Wolswijk, and M. Noble. 1992. In vitro analysis of the origin and maintenance of O-2Aadult progenitor cells. J Cell Biol 116: 167-76.

While the foregoing invention has been described in some detail for purposes of clarity and understanding, these particular embodiments and examples are to be considered as illustrative and not restrictive. It will be appreciated by one skilled in the art from a reading of this disclosure that various changes in form and detail can be made without departing from the true scope of the invention.

What is claimed:

1. A method for amplifying neural progenitor cells, the method comprising administering a growth factor to a brain progenitor cell in the presence of brain tissue, so as to obtain recruitment of neural progenitor cells, thereby amplifying neural progenitor cells.

2. The method of claim 1, further comprising recovering the neural progenitor cells from the brain tissue.

3. The method of claim 1, wherein the brain progenitor cell comprises an adult brain progenitor cell.

4. The method of claim 1, wherein the brain progenitor cell comprises a neonatal progenitor cell.

5. The method of claim 1, wherein the brain tissue comprises white matter.

6. The method of claim 1, wherein the brain tissue comprises a glial neoplasm.

7. The method of claim 1, wherein the growth factor comprises a PDGF, an FGF, a neuregulin, EGF, EGFR, or any combination thereof.

8. The method of claim 1, wherein the growth factor comprises PDGF-A.

9. The method of claim 1, wherein the growth factor comprises PDGF-B.

10. The method of claim 1, wherein the growth factor comprises FGF-2.

11. The method of claim 1, wherein the growth factor comprises GGF-2.

12. The method of claim 1, wherein the administering comprises introducing a replication incompetent retrovirus encoding the growth factor.

13. The method of claim 12, wherein the retrovirus is an amphoteric retrovirus.

14. The method of claim 1, wherein the administering comprises introducing a cell containing an isolated nucleotide sequence capable of expressing the growth factor, wherein the cell expresses the growth factor.

15. The method of claim 14, wherein the cell is a neural progenitor cell.

16. The method of claim 1, wherein the growth factor is linked to a reporter molecule.

17. The method of claim 1, wherein the growth factor is co-expressed with a reporter molecule.

18. The method of claim 17, wherein co-expression is achieved by an internal ribosome entry site (IRES).

19. The method of claim 16 or 17, wherein the reporter molecule comprises a fluorescent protein.

20. The method of claim 19, wherein the reporter molecule comprises green fluorescent protein, yellow fluorescent protein, blue fluorescent protein or DsRed.

21. The method of claim 1, wherein expression of the growth factor is inducible.

22. The method of claim 21, wherein the expression is induced by tetracycline.

23. The method of claim 21, wherein the expression is induced by tamoxifen.

24. A method for obtaining neural progenitor cells, the method comprising (a) infecting a brain progenitor cell with a replication incompetent retrovirus encoding a PDGF-B growth factor in the presence of brain tissue, and (b) recovering amplified neural progenitor cells from the brain tissue.

25. A method for amplification of resident progenitor cells in a tissue or organ, the method comprising administering a growth factor to the tissue or organ, so as to obtain recruitment of resident progenitor cells in the tissue or organ.

26. The method of claim 25, wherein the administration of the growth factor to the tissue or organ is in vivo.

27. The method of claim 25, wherein the administration of the growth factor to the tissue or organ is in vitro.

28. The method of claim 25, wherein the growth factor comprises a PDGF, an FGF, or a neuregulin, EGF, EGFR, or any combination thereof.

29. The method of claim 25, wherein the administering comprises introducing a nucleic acid encoding the growth factor.

30. The method of claim 25, wherein the growth factor is linked to a reporter molecule.

31. The method of claim 30, wherein the reporter molecule comprises a fluorescent protein.

32. The method of claim 31, wherein the reporter molecule comprises green fluorescent protein, yellow fluorescent protein, blue fluorescent protein or DsRed.

33. The method of claim 25, wherein growth factor expression is inducible.

34. The method of claim 33, wherein the expression is induced by tetracycline.

35. The method of claim 33, wherein the expression is induced by tamoxifen.

36. The method of claim 25, wherein the tissue or organ comprises brain, pancreas, liver, spinal cord, bone, heart, or kidney.

37. A method for treating a CNS injury or disease in a subject, the method comprising administering directly to the CNS of the subject a nucleic acid capable of expressing a growth factor, wherein expression of the growth factor causes recruitment of neural progenitor cells, thereby causing amplification of neural progenitor cells.

38. The method of claim 37, further comprising decreasing the expression of the growth factor, thereby decreasing the levels of growth factor in the brain, whereby the amplified neural progenitor cells will become differentiated.

39. The method of claim 37, further comprising
(a) recovering the neural progenitor cells from the subject;
(b) differentiating the neural progenitor cells in vitro; and
(c) returning the differentiated progenitor cells of step (b) to the CNS of the subject.

40. The method of claim 37, wherein the CNS injury or disease comprises Alzheimer's disease, multiple sclerosis, Parkinson's disease, Huntington's disease, stroke, dementia, trauma or any combination thereof.

41. The method of claim 37, wherein the growth factor comprises a PDGF, an FGF, a neuregulin, EGF, EGFR, or any combination thereof.

42. The method of claim 37, wherein the administering comprises introducing a cell containing a nucleic acid encoding the growth factor and capable of expressing the growth factor.

43. The method of claim 37, wherein the nucleic acid comprises an inducible promoter operably linked to a nucleic acid sequence encoding the growth factor.

44. The method of claim 39, wherein the recovering comprises cell sorting.

45. The method of claim 44, wherein the cell sorting comprises fluorescence activated cell sorting.

46. The method of claim 44, wherein the cell sorting comprises immunodetection of a progenitor cell surface molecule.

47. The method of claim 46, wherein the progenitor cell surface molecule is A2B5 surface ganglioside.

48. The method of claim 39, wherein the differentiating comprises culturing under conditions which induce the formation of astrocytes, oligodendrocytes, neurons, or any combination thereof.

49. A method for cell replacement therapy to treat an injury or disease in a subject, the method comprising
(a) administering one or more growth factors directly to a tissue of the subject, so as to obtain recruitment of resident progenitor cells in the tissue;
(b) recovering the progenitor cells from the subject;
(c) differentiating the progenitor cells in vitro; and
(d) returning the differentiated progenitor cells to the tissue of the subject.

50. The method of claim 49, wherein the administering comprises intralesional, intraperitoneal, intramuscular, intratumoral or intravenous injection; infusion; liposome- or vector-mediated delivery; or topical, nasal, oral, ocular, otic delivery, or any combination thereof.

51. The method of claim 49, wherein the administering is directly to the brain.

52. The method of claim 49, wherein the differentiating comprises defined cell culture conditions, genetic engineering of the cells, or a combination thereof.

53. The method of claim 49, wherein the tissue comprises brain, pancreas, liver, spinal cord, bone, heart, or kidney.

54. A genetically modified animal, wherein the animal's brain has been infected with one or more amphoteric viral vectors, and wherein at least one vector comprises a nucleic acid encoding PDGF-B and PDGF-B is expressed therefrom in the animal's brain, thereby forming a tumor in the animal's brain.

55. The animal of claim 54, wherein at least one vector comprises a nucleic acid encoding a reporter molecule and the reporter molecule is expressed therefrom in the animal's brain.

56. A method for determining whether a test compound is capable of treating a brain tumor, the method comprising:
(a) introducing one or more amphoteric viral vectors into an animal's brain, wherein at least one vector comprises a nucleic acid encoding PDGF-B and PDGF-B is expressed therefrom in the animal's brain, thereby forming a tumor in the animal's brain;
(b) administering an effective amount of the test compound to the animal of step (a);
(c) measuring the growth of the tumor in the animal of step (a); and
(d) comparing the measurement of tumor growth of step (b) to a measurement of tumor growth in an animal treated as in step (a) to which the test compound was not administered, wherein an arrest, delay, or reversal in tumor growth in the animal of step (a) indicates that the test compound is capable of treating a brain tumor.

57. A method for determining whether a test compound is capable of preventing a brain tumor, the method comprising:
(a) introducing one or more amphoteric viral vectors into an animal's brain, wherein at least one vector comprises a nucleic acid encoding PDGF-B and PDGF-B is expressed therefrom in the animal's brain;
(b) administering an effective amount of the test compound to the animal of step (a), wherein the administering occurs before formation of a tumor in the animal's brain;
(c) assessing brain tumor formation in the animal of step (a); and
(d) comparing the assessment of tumor formation of step (b) to a measurement of tumor formation in an animal treated as in step (a) to which the test compound was not administered, wherein an absence of tumor formation in the animal of step (a) indicates that the test compound is capable of preventing a brain tumor.

58. A method for determining brain tumor recurrence following brain tumor treatment, the method comprising:
(a) introducing one or more amphoteric viral vectors into an animal's brain, wherein at least one vector comprises a nucleic acid encoding PDGF-B and PDGF-B is expressed therefrom in the animal's brain, and wherein at least one vector comprises a nucleic acid encoding a reporter molecule and the reporter molecule is expressed therefrom in the animal's brain, thereby forming a tumor in the animal's brain;
(b) administering to the animal of step (a) one or more compounds in an effective amount to treat the tumor; and
(c) detecting expression of the reporter molecule in the brain of the animal of step (a), wherein detection of the reporter molecule indicates that the tumor will recur.

59. The method of claim 58, wherein PDGF-B and the reporter molecule are co-expressed from one vector.

60. The method of claim 59, wherein co-expression is achieved by an internal ribosome entry site (IRES).

61. The method of claim 58, wherein the reporter molecule comprises a fluorescent protein.

62. The method of claim 61, wherein the reporter molecule comprises green fluorescent protein, yellow fluorescent protein, blue fluorescent protein or DsRed.
